(19) United States
(12) Patent Application Publication
(10) Pub. No.: US 2025/0207107 A1
(43) Pub. Date: Jun. 26, 2025

(54) LUCIFERASE MUTANT AND USE THEREOF

(71) Applicant: DGI TECH (QING DAO) CO., LIMITED, Qingdao (CN)

(72) Inventors: Lulu PAN, Qingdao (CN); Ming NI, Qingdao (CN)

(73) Assignee: DGI TECH (QING DAO) CO., LIMITED, Qingdao (CN)

(21) Appl. No.: 19/005,452

(22) Filed: Dec. 30, 2024

Related U.S. Application Data
(63) Continuation of application No. PCT/CN2022/102827, filed on Jun. 30, 2022.

(51) Int. Cl. C12N 9/02; C12Q 1/66

(52) U.S. Cl. CPC C12N 9/0004; C12Q 1/66

(57) ABSTRACT

Provided are a luciferase mutant and the use thereof, and specifically provided are a copepod luciferase mutant and the use thereof. By means of performing protein directed evolution on Pxluc, a mutant which has more than 2-fold increased substrate specificity to ZS26/F-CTZ and a mutant which has more than 4-fold increased substrate specificity to ZS2/F-CTZ are obtained. The luciferase can be subjected to prokaryotic expression, the purification process is simple, and large-scale production is facilitated. The luciferase has similar luminescence to Gluc, is easy to detect, and has broad application prospects in the fields of basic scientific research, biological monitoring, biochemical diagnosis, etc.

### CROSS-REFERENCE TO RELATED APPLICATIONS

This application is a continuation of International Application No. PCT/CN2022/102827, filed on Jun. 30, 2022, the disclosure of which is hereby incorporated by reference in its entirety.

### STATEMENT REGARDING SEQUENCE LISTING

A Sequence Listing associated with this application is being filed concurrently herewith in XML format and is hereby incorporated by reference into the present specification. The XML file containing the Sequence listing is titled “980P010 Sequence Listing.xml”, was created on Mar. 19, 2025, and is 55,701 bytes in size.

### FIELD

The present disclosure relates to the field of biotechnology. In particular, the present disclosure relates to luciferase mutant and use thereof, particularly copepod luciferase mutant and use thereof.

### BACKGROUND

Luciferases are a class of enzymes capable of catalyzing the oxidative luminescence of luciferin or aliphatic aldehydes and widely exist in insects, bacteria, fungi, and marine organisms. Luciferases have become important tools in scientific research, and have been widely used in the fields of life science research, genome sequencing and analysis techniques, clinical medicine and forensic detection, drug screening, environmental monitoring, and enzyme-linked detection. Due to the characteristics of self-luminescence, luciferase is often used in the fields of living-cell detection, protein-protein interaction, protein localization, small interfering RNA silencing, and high-throughput drug screening. In the field of biological monitoring technology, luciferase can be used to detect the presence or absence of chemical contaminants, and it has broad application prospects in immunodetection and biochemical diagnosis. In addition, as a reporter gene to detect the expression intensity and transcriptional regulation of exogenous genes under different promoters, a combination of multiple luciferases with similar self-luminous brightness and different catalytic substrates is required.

At present, luciferase that have been well researched and developed mainly includes firefly luciferase (FLuc), bacterial luciferase (Lux), luciferase extracted from renilla (Renilla luciferase, RLuc), luciferase extracted from oplophorus (Oplophorus luciferase, OLuc), and luciferase extracted from marine animal Gaussia princeps (Gaussia luciferase, GLuc), etc. Fluc requires cofactors such as ATP, O₂ and Mg²⁺ and is expressed in a non-secretory way; Lux requires molecules including flavin mononucleotide (FMN), long-chain aliphatic aldehyde, oxygen and reduced nicotinamide adenine dinucleotide (NADH) for most of the fluorescent reactions; Rluc does not require ATP for Rluc luminescence, but it has week fluorescence intensity and cannot be expressed in a secretory way.

Although more than 40 bioluminescent systems have been found in nature, their exploitation is very limited.

### SUMMARY

The present disclosure is intended to solve at least one of the technical problems in the related art to some extent. To this end, an object of the present disclosure is to provide a mutant of copepoda luciferase (also known as Pleuromamma xiphia luciferase (Pxluc)). By means of performing protein-directed evolution on Pxluc, the inventors have obtained a mutant having more than 2-fold increased substrate specificity to ZS26/F-CTZ and a mutant having more than 4-fold increased substrate specificity to ZS2/F-CTZ. The luciferase can be expressed in prokaryotic and eukaryotic cells, and the purification process is simple and convenient for large-scale production. The luciferase has similar luminescence as Gluc, is easy to detect, and has broad application prospects in the fields of basic scientific research, biological monitoring, biochemical diagnosis, etc.

To this end, a first aspect of the present disclosure provides a mutated luciferase. According to an embodiment of the present disclosure, the mutated luciferase has at least one of the following mutation sites based on the amino acid sequence as set forth in SEQ ID NO: 2: sites 98, 99, 100, and 101, and the mutated luciferase includes or does not include a signal peptide amino acid sequence. According to an embodiment of the present disclosure, the mutated luciferase has a stronger catalytic activity for substrates such as coelenterazine, fluorinated coelenterazine, and coelenterazine derivatives. Compared with the existing Pleuromamma xiphia luciferase, the mutated luciferase has a wider substrate spectrum, a stronger substrate selection specificity, and a significantly enhanced luminescence. The mutated luciferase can be used in the fields of basic science research, biological detection technology, immunodetection, biochemical detection or diagnosis where the mutated luciferase are used for a luminescence detection, and thereby have a broad application prospect.

A second aspect of the present disclosure provides a nucleic acid molecule. According to an embodiment of the present disclosure, the nucleic acid molecule encodes the mutated luciferase of the first aspect. According to an embodiment of the present disclosure, the mutant (mutated luciferase) encoded by the nucleic acid molecule has a stronger catalytic activity for substrates such as coelenterazine, fluorinated coelenterazine, and coelenterazine derivatives. Compared with existing Pleuromamma xiphia luciferase, the mutant has a wider substrate spectrum or a stronger substrate selection specificity, and a significantly enhanced luminescence. The mutant can be used in the fields of basic science research, biological detection technology, immunodetection, biochemical detection or diagnosis where a protein is used for a luminescence detection, and thereby have a broad application prospect.

A third aspect of the present disclosure provides an expression vector. According to an embodiment of the present disclosure, the expression vector includes a nucleic acid molecule of the second aspect. The expression vector may include an optional control sequence, wherein the control sequence is operably linked to the nucleic acid molecule. The control sequence is one or more control sequences capable of directing expression of the nucleic acid molecule in a host. According to an embodiment of the present disclosure, the expression vector can efficiently express a protein in suitable host cells. The obtained protein has a stronger catalytic activity for substrates such as coelenterazine, fluorinated coelenterazine, and coelenterazine derivatives. Compared with the existing Pleuromamma xiphia luciferase, the obtained protein has a wider substrate spectrum or a stronger substrate selection specificity, and a significantly enhanced luminescence. The obtained protein can be used in the fields of basic science research, biological detection technology, immunodetection, biochemical detection or diagnosis where the protein is used for a luminescence detection, and thereby have a broad application prospect.

A fourth aspect of the present disclosure provides a recombinant cell. According to an embodiment of the present disclosure, the recombinant cell carries the nucleic acid molecule of the second aspect or the expression vector of the third aspect. The recombinant cell is obtained by transfecting or transforming the expression vector. According to an embodiment of the present disclosure, the recombinant cell can express the above-mentioned mutant with high efficiency under suitable conditions. The mutant has a stronger catalytic activity for substrates such as coelenterazine, fluorinated coelenterazine, and coelenterazine derivatives. Compared with the existing Pleuromamma xiphia luciferase, the mutant has a wider substrate spectrum or a stronger substrate selection specificity, and a significantly enhanced luminescence. The mutant can be used in the fields of basic science research, biological detection technology, immunodetection, biochemical detection or diagnosis where the protein is used for a luminescence detection, and thereby have a wide application prospect.

A fifth aspect of the present disclosure provides a method for producing a mutated luciferase. According to an embodiment of the present disclosure, the method includes: introducing the expression vector of the third aspect into recombinant cells; culturing and propagating the recombinant cells; and collecting product of said culturing and propagating, and extracting or purifying the mutated luciferase.

A sixth aspect of the present disclosure provides a method for detecting a nucleic acid sequence using the mutated luciferase of the first aspect. According to an embodiment of the present disclosure, the method for detecting a nucleic acid sequence includes the following steps: A) forming, by way of chemical coupling or bioconjugation or by means of fusion protein, a first mutated luciferase complex of a first specific recognition protein and the mutated luciferase of the first aspect; and forming, using a second luciferase as a signal peptide, a second mutated luciferase complex of a second specific recognition protein and the second luciferase by way of chemical coupling or bioconjugation or by means of fusion protein; B) reacting the first mutated luciferase complex with a first substrate to generate a first luminescent signal, and reacting the second luciferase complex with a second substrate to generate a second luminescent signal, wherein the first mutated luciferase complex and the second substrate do not have a significant cross-substrate reaction, and the second luciferase complex and the first substrate do not have a significant cross-substrate reaction, the first specific recognition protein recognizes and specifically binds to the first substrate, and the second specific recognition protein recognizes and specifically binds to the second substrate; and C) determining four bases A, T, G, and C for target nucleic acid sequencing by detecting fluorescence signal and signal combination of a self-luminescence system of the mutated luciferase and the second luciferase.

According to an embodiment of the present disclosure, when the mutated luciferase and the second luciferase are the same, the first substrate and the second substrate are the same; and when the mutated luciferase and the second luciferase are different, the first substrate and the second substrate are different.

According to another embodiment of the present disclosure, a method for detecting a nucleic acid sequence includes the following steps: 1) performing polymerization reaction between different bases labeled with affinity tags and having a reversible blocking modification and a template to be detected under the action of a polymerase; 2) adding a plurality of luciferase complexes in step A to couple the plurality of luciferase complexes to the different bases by specifically recognizing different affinity tags; 3) adding different substrates, determining types of the polymerized bases by detecting optical signals of the substrate or a combination thereof, and 4) adding a cleavage reagent to cleave the blocking group and linking group to prepare for the next round of polymerization.

A seventh aspect of the present disclosure provides a nucleic acid sequencing kit. According to an embodiment of the present disclosure, the kit includes a mutated luciferase of the first aspect or a luciferase complex.

An eighth aspect of the present disclosure provides a method for detecting the content of an analyte. According to an embodiment of the present disclosure, the method includes the following steps: a) forming, using the mutated luciferase of the first aspect as a signal peptide, a complex of a specific recognition protein of the analyte and the mutated luciferase by way of chemical coupling or bioconjugation or by means of fusion protein; b) contacting the analyte with the complex; c) adding a substrate for Pleuromamma xiphia luciferase or an analogue of the substrate to the reaction system; and d) determining the content of the analyte based on a fluorescence intensity of the reaction system detected subsequent to said adding the substrate for the Pleuromamma xiphia luciferase or the analogue of the substrate.

The mutant can be used as a signal protein for detecting the analyte based on the property that the mutant binds to and reacts with the substrate. For example, the mutant is coupled or fused with a protein capable of specifically recognizing the analyte by chemical coupling or forming a fusion protein; the protein capable of specifically recognizing the analyte can bind to the analyte; the mutant can catalyze the substrate thereof and perform self-luminescence; an intensity of bioluminescence released during the process of catalyzing the substrate by the mutant is measured by a chemiluminescent microplate reader; and the content of the analyte can be determined based on a level of the activity of the mutant, the activity of the mutant being reflected by the intensity of bioluminescence.

According to a specific embodiment of the present disclosure, the analyte may be a nucleic acid. The mutant provided in the present disclosure has a stronger catalytic activity for substrates such as coelenterazine, fluorinated coelenterazine, and coelenterazine derivatives. Compared with the existing Pleuromamma xiphia luciferase, the mutant has a wider substrate spectrum or a stronger substrate selection specificity, and a significantly enhanced luminescence. Therefore, the method can be used for detecting the nucleic acid expression of RNA or protein such as the expression amount of an RNA or a protein, and the localization or tracing of a protein. The method for nucleic acid detection has significantly higher accuracy and sensitivity than the existing Gaussian luciferase, and the obtained results are more accurate.

In a ninth aspect of the present disclosure, the present disclosure provides a method for screening a substrate for Pleuromamma xiphia luciferase. The method includes: I) contacting the mutated luciferase of the first aspect with a substrate to be screened to obtain a reaction mixture; and II) determining whether the substrate to be screened is a target substrate based on whether a chemical light signal is emitted by the reaction mixture obtained in step I). Based on the characteristic that the bioluminescence can be generated by binding the mutant to a target substrate, by contacting the substrate to be screened of interest with the mutant, the bioluminescence can be emitted in the process that the substrate to be screened is catalyzed by the mutant. By means of a chemiluminescence microplate reader, it is determined whether the substrate to be screened can be catalyzed by the mutant to emit chemiluminescence, thereby determining whether the substrate to be screened is the target substrate. Therefore, the target substrate can be accurately screened out with the method of an embodiment of the present disclosure.

It is to be understood that, without departing from the scope of the present disclosure, the respective above-mentioned features of the present disclosure and the respective features specifically described below (for example, those described in the embodiments) may be combined with each other to constitute new or preferred embodiments, which are not elaborated due to the length limitation.

### BRIEF DESCRIPTION OF THE DRAWINGS

The foregoing and/or additional aspects and advantages of the present disclosure will become apparent and readily appreciated from the following description of the embodiments, in conjunction with the accompanying drawings.

FIG. 1 illustrates a plasmid profile of the wild type Pleuromamma xiphia luciferase prokaryotic expression plasmid pET28a-Pxluc WT containing a signal peptide;

FIG. 2 illustrates a plasmid profile of the wild type Pleuromamma xiphia luciferase prokaryotic expression plasmid pCold WT Pxluc containing a signal peptide;

FIG. 3 illustrates a plasmid profile of the wild type Pleuromamma xiphia luciferase prokaryotic expression plasmid pCold WT NS Pxluc without a signal peptide;

FIG. 4 is a graph showing the results of expression and purification of wild type Pleuromamma xiphia luciferase in different competent cells, with the arrows indicating bands of the protein of interest;

FIG. 5 illustrates chemical structures of substrate A (coelenterazine), substrate B (fluorinated coelenterazine), substrate C (coelenterazine derivative ZS2), and substrate D (coelenterazine derivative ZS26);

FIG. 6 illustrates results of protein level activity detection of Pleuromamma xiphia luciferase and Gaussian luciferase expressed in prokaryotic cells;

FIG. 7 illustrates results of protein level activity detection of the dominant mutant of Pleuromamma xiphia luciferase on the specific substrate ZS26/F-CTZ;

FIG. 8 illustrates results of protein level activity detection of the dominant mutant of Pleuromamma xiphia luciferase on the specific substrate ZS2/F-CTZ;

FIG. 9 illustrates a plasmid profile of the wild type Pleuromamma xiphia luciferase eukaryotic expression plasmid pEE12.4-Pxluc WT containing a signal peptide;

FIG. 10 is a graph showing the results of the expression and purification of Pleuromamma xiphia luciferase in eukaryotic cells, with the arrows indicating bands of the protein of interest;

FIG. 11 is a graph showing the results before and after purification of Pleuromamma xiphia luciferase-coupled SA protein; and

FIG. 12 illustrates results of protein level activity detection of Xiphophorus luciferase pEE12.4 Pxluc WT, mutant pEE12.4 Pxluc P26-95, coupled SA-Pxluc WT and SA Pxluc P26-95, expressed in eukaryotic cells, on the substrate-specific ZS26/F-CTZ and ZS2/F-CTZ.

### DETAILED DESCRIPTION

Embodiments of the present disclosure are described in detail below. Examples of the embodiments are illustrated in the accompanying drawings. The embodiments described below with reference to the accompanying drawing are illustrative for explaining the present disclosure, rather than being construed as limitations of the present disclosure.

Furthermore, the terms “first” and “second” are used for descriptive purposes only and are not to be construed as indicating or implying relative importance or implicitly indicating the number of technical features indicated. In this regard, a feature defined as “first” or “second” may explicitly or implicitly include at least one such feature. In the description of the present disclosure, the meaning of “plurality of” is at least two, e.g. two, three, etc., unless specifically and specifically limited otherwise.

In the field of basic research, the luciferase gene has been widely used as a reporter gene to study the expression intensity and transcriptional regulation of exogenous genes under different promoters. In the field of biological monitoring technology, luciferase can be used to detect the presence or absence of chemical contaminants. In addition, it has broad application prospects in immunodetection and biochemical diagnosis.

### Mutant

In an aspect of the present disclosure, the present disclosure provides a luciferase mutant having one or more of the following mutation sites based on the amino acid sequence as set forth in SEQ ID NO: 2: sites 98, 99, 100, and 101. The luciferase mutant includes or does not include a signal peptide amino acid sequence.

The mutants obtained by modifying the above-mentioned amino acid sequence at the mutation sites according to an embodiment of the present disclosure have a stronger catalytic activity for substrates such as coelenterazine, fluorinated coelenterazine, and coelenterazine derivatives ZS2 and ZS26. Compared with the existing Pleuromamma xiphia luciferase, the mutants have a wider substrate spectrum, a stronger specificity, and a significantly enhanced luminescence. The mutants can be used in the fields of basic science research, biological detection technology, immunodetection, biochemical detection or diagnosis where the mutant is used for a luminescence detection, and thereby have a broad application prospect. For example, the mutants can be used as a reporter gene for quantitative detection of DNA, RNA, transcription factors, proteins or cells, etc. The mutants can be used as a luminescence signal protein in fusion protein to quantitatively detect target small molecules, proteins, etc.

As used herein, the term “reporter gene” is a molecular biological concept referring to a type of genes that are expressed in cells, tissues/organs, or individual under specific conditions and that can produce a trait, which is readily detectable and the test material would not otherwise produce, i.e., a gene encoding a detectable protein or enzyme. A reporter gene must satisfy the following conditions in terms of genetic selection and screening detection: 1. the reporter gene has been cloned and the complete sequence thereof has been determined; 2. the expression product is not present in the recipient cells, i.e. no background, and no similar endogenous expression product exists in the transfected cells; 3. the expression product thereof can be quantitatively determined. The reporter gene can be used in the following manners, but not limited to the following manners: fusing a reporter gene with a gene expression regulatory sequence to form a chimeric gene, or fusing a reporter gene with other genes of interest, allowing the nucleic acid to express under the control of the regulatory sequence, detecting the expression regulation of the gene of interest based on the expression product thereof, and studying the nucleic acid.

As used herein, the term “nucleic acid expression” may refer to the expression of DNA as RNA, or the expression of DNA as a protein via RNA, or the expression of RNA as a protein. That is, the product obtained after expression of the nucleic acid herein can be either an RNA or a protein.

As used herein, “chemiluminescence”, also referred to as cold light, is optical radiation produced by a chemical reaction in the absence of any excitation by light, heat, electric field, or the like. Chemiluminescence is also found in living systems, referred to as bioluminescence, e.g., light emitted by firefly, certain bacteria or fungi, protozoa, helminths, and crustaceans. In the present disclosure, the mutants may undergo self-luminescence, i.e., chemiluminescence.

According to some specific embodiments of the present disclosure, the above-mentioned mutant may further include at least one of the following additional technical features.

According to some specific embodiments of the present disclosure, the gene sequence of wild type Pleuromamma xiphia luciferase (WT Pxluc) containing a signal peptide is as set forth in SEQ ID NO: 1, and the encoded amino acid thereof is as set forth in SEQ ID NO: 2, in which amino acids at sites 1 to 17 are the signal peptide (in bold); and the gene sequence of the wild type Pleuromamma xiphia luciferase without a signal peptide (WT no signal peptide Pxluc: WT-NS Pxluc) is as set forth in SEQ ID NO: 3, and the encoded amino acid thereof is as set forth in SEQ ID NO: 4. The sequences as set forth in SEQ ID NO: 1 to SEQ ID NO: 4 are as follows:

(SEQ ID NO: 1)
ATGTATATAAAAGTTTGGTTTGGTCTGGCTTGTCTTTCATTGGTTCTGGC
CCAACCAACTGAAAACAAGCAGGAGTCTCATATTGTAGATTCAGATCTTG
ATGGCGACCGTGGTAGGAAGTTGCCCGGAAAAAAGCTTCCTATAGAAGTA
CTCAAAATCATGGAAGCCAATGCCAGGAGAGCTGGTTGCACTAGAGGATG
TCTCATATGTCTTTCAAAAATCAAGTGTACAGCCAAAATGAAGCGATACA
TTCCAGGGAGATGTCATACTTATGAAGGAGATAAATCTATTGGACAGGGA
GGCATAGGTGGCCCTATTGTTGATATTCCTGAAATTATTGGATTCAAGAA
CATGGAACCCATGGATCAGTTCATCGCACAAGTTGATCTGTGCGCCGACT
GTACAACTGGGTGCCTGAAAGGCCTTGCTAATGTTAGGTGCAATGACTTG
CTGAAGAAATGGCTGCCTGACAGATGTGCTGGTTTTGCCGACAAAATTCA
AAATGAAGTGGATAGTATCAAGGGCATGGCTGGGGATCGC
(SEQ ID NO: 2)
MYIKVWFGLACLSLVLAQPTENKQESHIVDSDLDGDRGRKLPGKKLPIEV
LKIMEANARRAGCTRGCLICLSKIKCTAKMKRYIPGRCHTYEGDKSIGQG
GIGGPIVDIPEIIGFKNMEPMDQFIAQVDLCADCTTGCLKGLANVRCNDL
LKKWLPDRCAGFADKIQNEVDSIKGMAGDR
(SEQ ID NO: 3)
CAACCAACTGAAAACAAGCAGGAGTCTCATATTGTAGATTCAGATCTTGA
TGGCGACCGTGGTAGGAAGTTGCCCGGAAAAAAGCTTCCTATAGAAGTAC
TCAAAATCATGGAAGCCAATGCCAGGAGAGCTGGTTGCACTAGAGGATGT
CTCATATGTCTTTCAAAAATCAAGTGTACAGCCAAAATGAAGCGATACAT
TCCAGGGAGATGTCATACTTATGAAGGAGATAAATCTATTGGACAGGGAG
GCATAGGTGGCCCTATTGTTGATATTCCTGAAATTATTGGATTCAAGAAC
ATGGAACCCATGGATCAGTTCATCGCACAAGTTGATCTGTGCGCCGACTG
TACAACTGGGTGCCTGAAAGGCCTTGCTAATGTTAGGTGCAATGACTTGC
TGAAGAAATGGCTGCCTGACAGATGTGCTGGTTTTGCCGACAAAATTCAA
AATGAAGTGGATAGTATCAAGGGCATGGCTGGGGATCGC
(SEQ ID NO: 4)
QPTENKQESHIVDSDLDGDRGRKLPGKKLPIEVLKIMEANARRAGCTRGC
LICLSKIKCTAKMKRYIPGRCHTYEGDKSIGQGGIGGPIVDIPEIIGFKN
MEPMDQFIAQVDLCADCTTGCLKGLANVRCNDLLKKWLPDRCAGFADKIQ
NEVDSIKGMAGDR

According to some specific embodiments of the present disclosure, the mutant has one or more of the following mutations (1) to (4) based on the amino acid sequence as set forth in SEQ ID NO: 2:
(1) G at site 98 is mutated to L, P, Q, S, or T;
(2) Q at site 99 is mutated to R, W, I, Y, A, L, F, V, P, E, or M;
(3) G at site 100 is mutated to S, Q, R, W, T, A, or L; and
(4) G at site 101 is mutated to F, R, S, C, Y, L, I, K, V, or P.

According to specific embodiments of the present disclosure, the mutated luciferase has one or two of the following mutation sites based on the amino acid sequence as set forth in SEQ ID NO: 2: sites 98, 99, 100 and 101, and the mutated luciferase includes or does not include a signal peptide amino acid sequence.

According to further specific embodiments of the present disclosure, the mutant has one or two of the following mutations (1) to (4) based on the amino acid sequence as set forth in SEQ ID NO: 2:
(1) G at site 98 is mutated to L, P, Q, S, or T;
(2) Q at site 99 is mutated to R, W, I, Y, A, L, F, V, P, E, or M;
(3) G at site 100 is mutated to S, Q, R, W, T, A, or L; and
(4) G at site 101 is mutated to F, R, S, C, Y, L, I, K, V, or P.

According to some specific embodiments of the present disclosure, the mutant has the following mutations based on the amino acid sequence as set forth in SEQ ID NO: 2:
1) G at site 98 is mutated to L, and Q at site 99 is mutated to R; or
2) G at site 98 is mutated to P; or
3) G at site 98 is mutated to Q; or
4) G at site 98 is mutated to S, and Q at site 99 is mutated to W; or
5) Q at site 98 is mutated to I; or
6) Q at site 99 is mutated to Y; or
7) Q at site 99 is mutated to A; or
8) Q at site 99 is mutated to L; or
9) Q at site 99 is mutated to F; or
10) G at site 98 is mutated to L, and Q at site 99 is mutated to V; or
11) G at site 98 is mutated to T, and Q at site 99 is mutated to P; or
12) G at site 98 is mutated to L, and Q at site 99 is mutated to E; or
13) Q at site 99 is mutated to M, and G at site 100 is mutated to S; or
14) G at site 100 is mutated to Q, and G at site 101 is mutated to F; or
15) G at site 100 is mutated to R, and G at site 101 is mutated to R; or
16) G at site 100 is mutated to W, and G at site 101 is mutated to F; or
17) G at site 100 is mutated to S; or
18) G at site 100 is mutated to T, and G at site 101 is mutated to S; or
19) G at site 100 is mutated to R, and G at site 101 is mutated to C; or
20) G at site 100 is mutated to A, and G at site 101 is mutated to R; or
21) G at site 100 is mutated to L, and G at site 101 is mutated to Y; or
22) G at site 100 is mutated to S, and G at site 101 is mutated to L; or
23) G mutation at site 101 is I; or
24) G at site 100 is mutated to L, and G at site 101 is mutated to K; or
25) G at site 100 is mutated to T; or
26) G at site 100 is mutated to A, and G at site 101 is mutated to V; or
27) G at site 100 is mutated to A, and G at site 101 is mutated to P.

According to some specific embodiments of the present disclosure, when the amino acid sequence as set forth in SEQ ID NO: 2 has the above-mentioned mutation, the obtained protein has a relatively stronger catalytic activity for substrates such as coelenterazine, fluorinated coelenterazine, and coelenterazine derivatives. Compared with the existing Pleuromamma xiphia luciferase, the protein has a wider substrate spectrum and significantly enhanced luminescence. The protein has a significantly improved detection accuracy in practical applications, and can be used in the fields of basic science research, biological detection technology, immunodetection, biochemical detection or diagnosis where the protein is used for a luminescence detection, and thereby has a broad application prospect.

According to some specific embodiments of the present disclosure, the mutated luciferase does not contain a signal peptide sequence.

According to some specific embodiments of the present disclosure, the mutated luciferase may be a luciferase having a mutation at one or more of sites 98, 99, 100, and 101 based on the amino acid sequence as set forth in SEQ ID NO: 2 (e.g., the mutation may be at any one of the four mutation sites, or at two of the four mutation sites in combination, or at three of the four mutation sites in combination, or at all of the four mutation sites).

According to some specific embodiments of the present disclosure, the mutated luciferase may also have one or more of the mutation sites 98, 99, 100, and 101 based on the amino acid sequence as set forth in SEQ ID NO: 2, and the mutated luciferase does not contain a signal peptide amino acid sequence.

The amino acids at sites 1 to 17 of the amino acid sequence as set forth in SEQ ID NO: 2 are a signal peptide. The inventors found that, as long as one or more of sites 98, 99, 100, and 101 (these sites are in the SEQ ID NO: 2 sequence) are mutated, the mutated luciferase obtained based on either the complete sequence of SEQ ID NO: 2 containing the signal peptide or the sequence without the signal peptide can have a stronger catalytic activity for substrates such as coelenterazine, fluorinated coelenterazine, and coelenterazine derivative ZS2, and has a wider substrate spectrum, a stronger specificity, and a significantly enhanced luminescence compared with the existing Pleuromamma xiphia luciferase. According to some specific embodiments of the present disclosure, the mutant is a non-secreted protein or a secreted protein.

According to some specific embodiments of the present disclosure, the present disclosure provides a nucleic acid molecule encoding the above-mentioned mutant.

It should be noted that, with respect to the nucleic acid mentioned in the present specification and claims, those skilled in the art can understand that the nucleic acid includes any one of or both of complementary duplexes. For convenience, in this specification and claims, while in most cases only one strand is shown, in fact, the other strand complementary thereto is also disclosed. In addition, the nucleic acid sequences of the present disclosure include DNA forms or RNA forms, one of which is disclosed, meaning that the other is also disclosed.

In another aspect of the present disclosure, the present disclosure provides an expression vector including the above-mentioned nucleic acid molecule. The expression vector herein is not limited a specific type, as long as the corresponding mutant is capable of replicative expression in a host cell. The expression vector may include an optional control sequence, wherein the control sequence is operably linked to the nucleic acid molecule. The control sequence is one or more control sequences capable of directing expression of the nucleic acid molecule in a host. According to some specific embodiments of the present disclosure, the expression vector can efficiently express a protein in a suitable host cell, and the obtained protein has a stronger catalytic activity for substrates such as coelenterazine, fluorinated coelenterazine, and coelenterazine derivatives. Compared with the existing Pleuromamma xiphia luciferase, the protein has a wider substrate spectrum, a stronger specificity, and a significantly enhanced luminescence. The obtained protein can be used in the fields of basic science research, biological detection technology, immunodetection, biochemical detection or diagnosis where the protein is used for a luminescence detection, and thereby have a broad application prospect.

In a further aspect, the present disclosure provides a recombinant cell carrying a nucleic acid molecule, expression vector, or mutant as described above. The recombinant cell is obtained by transfecting or transforming the expression vector. According to some specific embodiments of the present disclosure, the recombinant cell can express the above-mentioned mutant under suitable conditions with high efficiency. The mutant has a stronger catalytic activity for substrates such as coelenterazine, fluorinated coelenterazine and coelenterazine derivatives. Compared with the existing Pleuromamma xiphia luciferase, the mutant has a wider substrate spectrum, a stronger specificity and a significantly enhanced luminescence. The mutant can be used in the fields of basic science research, biological detection technology, immunodetection, biochemical detection or diagnosis where the protein is used for a luminescence detection, and thereby have a broad application prospect.

According to some specific embodiments of the present disclosure, the above-mentioned recombinant cell may further include at least one of the following additional technical features.

According to some specific embodiments of the present disclosure, the recombinant cell is Escherichia coli, yeast, or mammalian cell. According to some specific embodiments of the present disclosure, the recombinant cell is not specifically limited. Any cell capable of expressing the mutant in a nucleic acid or vector (e.g., plasmid), for example, yeast cells, bacteria, or mammalian cells such as human embryonic kidney cells, can be used as the recombinant cells.

According to some specific embodiments of the present disclosure, the recombinant cells do not include animal germ cells, fertilized eggs, or embryonic stem cells.

According to some specific embodiments of the present disclosure, the present disclosure provides a method for producing a mutated luciferase. The method includes: introducing the expression vector described above into recombinant cells; culturing and propagating the recombinant cells; and collecting product of said culturing and propagating, and extracting or purifying the mutated luciferase. According to some specific embodiments of the present disclosure, the present disclosure provides the use of the mutated luciferase as described above and a corresponding substrate in the detection of a nucleic acid sequence.

It should be noted that in the present disclosure, “mutated luciferase”, “luciferase mutant”, and “mutant” can be equivalently replaced with each other.

### Kit and Use for Prepared Kit

In an aspect of the present disclosure, the present disclosure provides a kit for detecting a content of an analyte. The kit includes the luciferase mutant as described above. A specific recognition protein of the analyte is suitable for forming a complex with the mutant.

According to some specific embodiments of the present disclosure, the kit further includes a substrate for Pleuromamma xiphia luciferase or an analogue of the substrate.

According to some specific embodiments of the present disclosure, the substrate is selected from at least one of coelenterazine, fluorinated coelenterazine, or coelenterazine derivative.

According to some specific embodiments of the present disclosure, coelenterazine derivative is selected from coelenterazine derivative ZS2 or coelenterazine derivative ZS26.

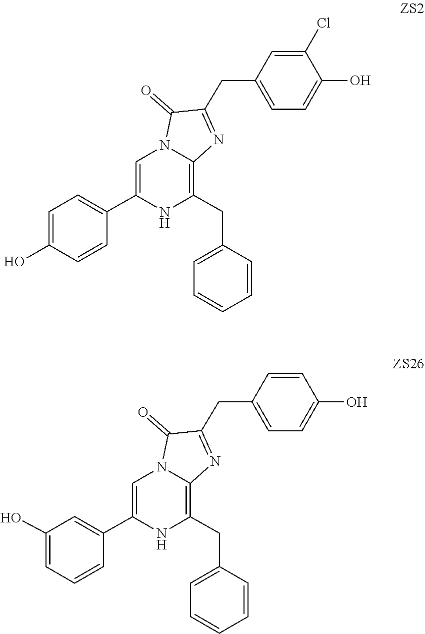 [SMILES: O=c1c(Cc2ccc(O)c(Cl)c2)nc2c(Cc3ccccc3)[nH]c(-c3ccc(O)cc3)cn1-2 ; O=c1c(Cc2ccc(O)cc2)nc2c(Cc3ccccc3)[nH]c(-c3cccc(O)c3)cn1-2]

According to some specific embodiments of the present disclosure, the kit further includes a specific recognition protein for the analyte.

The present disclosure provides a nucleic acid sequencing kit. The kit includes the mutated luciferase as described above.

In a further aspect of the present disclosure, the present disclosure provides the use of the mutant as described above in the preparation of a kit for detecting a content of an analyte, and a specific recognition protein of the analyte is suitable for forming a complex with the mutant. Based on the characteristic that the mutant reacts with the substrate, the mutant can be used as a signal protein to detect the analyte. For example, by means of chemical coupling or forming a fusion protein, the mutant is coupled or fused with a protein capable of specifically recognizing the analyte. When the analyte, the mutant, and the substrate are in the same system, the protein capable of specifically recognizing the analyte can bind to the analyte. The mutant can catalyze the substrate thereof and perform self-luminescence. The intensity of the bioluminescence released by the mutant during the process of catalyzing the substrate thereof is determined by a chemiluminescent microplate reader. The intensity of the bioluminescence can reflect the activity of the mutant, and the content of the analyte can be determined based on the level of the activity. Before the substrate binds to the mutant, it is necessary to wash off the non-specific binding protein in the system, to exclude the interference of other factors on the detection result. Thus, the mutant can be used to prepare a kit and accurately detect the content of the analyte.

According to some specific embodiments of the present disclosure, the above-mentioned use may further include at least one of the following additional technical features.

According to some specific embodiments of the present disclosure, the kit further includes a substrate for Pleuromamma xiphia luciferase or an analogue of the substrate.

According to some specific embodiments of the present disclosure, the substrate is selected from at least one of coelenterazine, fluorinated coelenterazine, or coelenterazine derivative.

According to some specific embodiments of the present disclosure, the substrate is not specifically limited. The substances that can chemically react with the mutant shall fall within the scope of the substrate (but not limited to chemiluminescence reaction). A person skilled in the art can change different substrates according to experimental requirements.

According to some specific embodiments of the present disclosure, coelenterazine derivative is selected from coelenterazine derivative ZS2 or coelenterazine derivative ZS26.

According to some specific embodiments of the present disclosure, the kit further includes a specific recognition protein for the analyte.

### Methods

In an aspect of the present disclosure, the present disclosure provides a method for detecting a content of a substance. The method includes the following steps:
i) forming a complex between the analyte-specific recognition protein and the mutant as described above;
ii) contacting an analyte with the complex;
iii) adding a substrate for Pleuromamma xiphia luciferase or an analogue of the substrate to the reaction system;
iv) determining a content of an analyte based on fluorescence intensity of the reaction system subsequent to said adding the substrate for Pleuromamma xiphia luciferase or the analogue of the substrate.

According to some specific embodiments of the present disclosure, the present disclosure provides a method for detecting a content of an analyte. The method includes the following steps:
a) forming, using the above-mentioned mutated luciferase as a signal protein, a complex of a specific recognition protein of the analyte and the mutated luciferase by way of chemical coupling or bioconjugation or by means of fusion protein;
b) contacting the analyte with the complex;
c) adding a substrate for Pleuromamma xiphia luciferase or an analogue of the substrate to the reaction system;
d) determining the content of the analyte based on a fluorescence intensity of the reaction system detected subsequent to said adding the substrate for the Pleuromamma xiphia luciferase or the analogue of the substrate.

According to some specific embodiments of the present disclosure, the substrate is selected from at least one of coelenterazine, fluorinated coelenterazine, or coelenterazine derivative.

According to some specific embodiments of the present disclosure, coelenterazine derivative is selected from coelenterazine derivative ZS2 or coelenterazine derivative ZS26.

In yet another aspect of the present disclosure, the present disclosure provides a method for screening a substrate for Pleuromamma xiphia luciferase. The method includes:
I) contacting the above-mentioned mutant with a substrate to be screened to obtain a reaction mixture; and
II) determining, based on whether the reaction mixture obtained in step I) emits a chemical light signal, whether the substrate to be screened is a target substrate.

According to some specific embodiments of the present disclosure, the reaction mixture obtained in step I) emits a chemical light signal indicative that the substrate to be screened is a target substrate.

The present disclosure is described with reference to specific examples. These examples are intended to be illustrative only and not limiting in any way.

### Example 1: Design and Construction of Plasmid for Wild-Type Pleuromamma xiphia Luciferase to be Expressed in Prokaryotic Cells

In the present example, wild type Pleuromamma xiphia luciferase with and without a signal peptide were constructed to evaluate the effects of the signal peptide on the wild type Pleuromamma xiphia luciferase.

The gene sequence of the wild type Pleuromamma xiphia luciferase (WT Pxluc) containing a signal peptide is as set forth in SEQ ID NO: 1, and the encoded amino acid thereof is as set forth in SEQ ID NO: 2, in which amino acids at sites 1 to 17 are the signal peptide (in bold); and the gene sequence of the wild type Pleuromamma xiphia luciferase without a signal peptide (WT no signal peptide Pxluc: WT-NS Pxluc) is as set forth in SEQ ID NO: 3, and the encoded amino acid thereof is as set forth in SEQ ID NO: 4.

The gene sequence, as set forth in SEQ ID NO: 5, of Pleuromamma xiphia luciferase containing a signal peptide for pET28a vector (pET28a Pxluc WT) was synthesized by means of total gene synthesis technology (Sangon Biotech). The plasmid profile is illustrated in FIG. 1, which was fused with a purification tag containing six histidine (6×His) at the C-terminal to facilitate protein purification. The cleavage sites at both ends were BamHI and NotI.

The gene sequence, as set forth in SEQ ID NO: 6, of Pleuromamma xiphia luciferase containing a signal peptide for pCold vector was synthesized by means of PCR technology and fused with a purification tag containing six histidine (6×His) at the C-terminal to facilitate protein purification. The gene sequence, as set forth in SEQ ID NO: 7, of Pleuromamma xiphia luciferase without the signal peptide for pCold vector was synthesized by means of PCR technology and fused with a purification tag containing six histidine (6×His) at the C-terminal to facilitate protein purification.

(SEQ ID NO: 1)
ATGTATATAAAAGTTTGGTTTGGTCTGGCTTGTCTTTCATTGGTTCTGGC
CCAACCAACTGAAAACAAGCAGGAGTCTCATATTGTAGATTCAGATCTTG
ATGGCGACCGTGGTAGGAAGTTGCCCGGAAAAAAGCTTCCTATAGAAGTA
CTCAAAATCATGGAAGCCAATGCCAGGAGAGCTGGTTGCACTAGAGGATG
TCTCATATGTCTTTCAAAAATCAAGTGTACAGCCAAAATGAAGCGATACA
TTCCAGGGAGATGTCATACTTATGAAGGAGATAAATCTATTGGACAGGGA
GGCATAGGTGGCCCTATTGTTGATATTCCTGAAATTATTGGATTCAAGAA
CATGGAACCCATGGATCAGTTCATCGCACAAGTTGATCTGTGCGCCGACT
GTACAACTGGGTGCCTGAAAGGCCTTGCTAATGTTAGGTGCAATGACTTG
CTGAAGAAATGGCTGCCTGACAGATGTGCTGGTTTTGCCGACAAAATTCA
AAATGAAGTGGATAGTATCAAGGGCATGGCTGGGGATCGC
(SEQ ID NO: 2)
MYIKVWFGLACLSLVLAQPTENKQESHIVDSDLDGDRGRKLPGKKLPIEV
LKIMEANARRAGCTRGCLICLSKIKCTAKMKRYIPGRCHTYEGDKSIGQG
GIGGPIVDIPEIIGFKNMEPMDQFIAQVDLCADCTTGCLKGLANVRCNDL
LKKWLPDRCAGFADKIQNEVDSIKGMAGDR
(SEQ ID NO: 3)
CAACCAACTGAAAACAAGCAGGAGTCTCATATTGTAGATTCAGATCTTGA
TGGCGACCGTGGTAGGAAGTTGCCCGGAAAAAAGCTTCCTATAGAAGTAC
TCAAAATCATGGAAGCCAATGCCAGGAGAGCTGGTTGCACTAGAGGATGT
CTCATATGTCTTTCAAAAATCAAGTGTACAGCCAAAATGAAGCGATACAT
TCCAGGGAGATGTCATACTTATGAAGGAGATAAATCTATTGGACAGGGAG
GCATAGGTGGCCCTATTGTTGATATTCCTGAAATTATTGGATTCAAGAAC
ATGGAACCCATGGATCAGTTCATCGCACAAGTTGATCTGTGCGCCGACTG
TACAACTGGGTGCCTGAAAGGCCTTGCTAATGTTAGGTGCAATGACTTGC
TGAAGAAATGGCTGCCTGACAGATGTGCTGGTTTTGCCGACAAAATTCAA
AATGAAGTGGATAGTATCAAGGGCATGGCTGGGGATCGC
(SEQ ID NO: 4)
QPTENKQESHIVDSDLDGDRGRKLPGKKLPIEVLKIMEANARRAGCTRGC
LICLSKIKCTAKMKRYIPGRCHTYEGDKSIGQGGIGGPIVDIPEIIGFKN
MEPMDQFIAQVDLCADCTTGCLKGLANVRCNDLLKKWLPDRCAGFADKIQ
NEVDSIKGMAGDR
(SEQ ID NO: 5)
aagcttgccgccaccATGTATATAAAAGTTTGGTTTGGTCTGGCTTGTCT
TTCATTGGTTCTGGCCCAACCAACTGAAAACAAGCAGGAGTCTCATATTG
TAGATTCAGATCTTGATGGCGACCGTGGTAGGAAGTTGCCCGGAAAAAAG
CTTCCTATAGAAGTACTCAAAATCATGGAAGCCAATGCCAGGAGAGCTGG
TTGCACTAGAGGATGTCTCATATGTCTTTCAAAAATCAAGTGTACAGCCA
AAATGAAGCGATACATTCCAGGGAGATGTCATACTTATGAAGGAGATAAA
TCTATTGGACAGGGAGGCATAGGTGGCCCTATTGTTGATATTCCTGAAAT
TATTGGATTCAAGAACATGGAACCCATGGATCAGTTCATCGCACAAGTTG
ATCTGTGCGCCGACTGTACAACTGGGTGCCTGAAAGGCCTTGCTAATGTT
AGGTGCAATGACTTGCTGAAGAAATGGCTGCCTGACAGATGTGCTGGTTT
TGCCGACAAAATTCAAAATGAAGTGGATAGTATCAAGGGCATGGCTGGGG
ATCGCggtggcagtgaaaatctgtattttcagagcggcggccatcatcat
catcatcatggcagctgatgagaattc
(SEQ ID NO: 6)
caccatgaatcacaaagtgcatatgATGTATATAAAAGTTTGGTTTGGTC
TGGCTTGTCTTTCATTGGTTCTGGCCCAACCAACTGAAAACAAGCAGGAG
TCTCATATTGTAGATTCAGATCTTGATGGCGACCGTGGTAGGAAGTTGCC
CGGAAAAAAGCTTCCTATAGAAGTACTCAAAATCATGGAAGCCAATGCCA
GGAGAGCTGGTTGCACTAGAGGATGTCTCATATGTCTTTCAAAAATCAAG
TGTACAGCCAAAATGAAGCGATACATTCCAGGGAGATGTCATACTTATGA
AGGAGATAAATCTATTGGACAGGGAGGCATAGGTGGCCCTATTGTTGATA
TTCCTGAAATTATTGGATTCAAGAACATGGAACCCATGGATCAGTTCATC
GCACAAGTTGATCTGTGCGCCGACTGTACAACTGGGTGCCTGAAAGGCCT
TGCTAATGTTAGGTGCAATGACTTGCTGAAGAAATGGCTGCCTGACAGAT
GTGCTGGTTTTGCCGACAAAATTCAAAATGAAGTGGATAGTATCAAGGGC
ATGGCTGGGGATCGCggtggcagtgagaacctgtac
(SEQ ID NO: 7)
catgaatcacaaagtgcatatgCAACCAACTGAAAACAAGCAGGAGTCTC
ATATTGTAGATTCAGATCTTGATGGCGACCGTGGTAGGAAGTTGCCCGGA
AAAAAGCTTCCTATAGAAGTACTCAAAATCATGGAAGCCAATGCCAGGAG
AGCTGGTTGCACTAGAGGATGTCTCATATGTCTTTCAAAAATCAAGTGTA
CAGCCAAAATGAAGCGATACATTCCAGGGAGATGTCATACTTATGAAGGA
GATAAATCTATTGGACAGGGAGGCATAGGTGGCCCTATTGTTGATATTCC
TGAAATTATTGGATTCAAGAACATGGAACCCATGGATCAGTTCATCGCAC
AAGTTGATCTGTGCGCCGACTGTACAACTGGGTGCCTGAAAGGCCTTGCT
AATGTTAGGTGCAATGACTTGCTGAAGAAATGGCTGCCTGACAGATGTGC
TGGTTTTGCCGACAAAATTCAAAATGAAGTGGATAGTATCAAGGGCATGG
CTGGGGATCGCggtggcagtgagaacctgta

### 1. Obtaining Inserted Fragment

Whole gene synthetic wild type Pleuromamma xiphia luciferase containing a signal peptide (pET28a Pxluc WT) was dissolved in deionized water and diluted to a concentration of 10 ng/l, as template DNA. By using the KOD FXD Neo enzyme, PCR reaction system was prepared and PCR reaction was performed according to the manual thereof, to prepare the inserted fragment.

Table 1 lists the sequences of primers used for the PCR reaction of the wild type Pleuromamma xiphia luciferase containing a signal peptide (WT-Pxluc) and the wild type Pleuromamma xiphia luciferase without a signal peptide (WT-NS Pxluc). The PCR reaction system is shown in Table 2. The reaction conditions are shown in Table 3, and the number of PCR cycles is 30.

TABLE 1

| Name | Sequence |
|---|---|
| Forward Primer | 5′-CACCATGAATCACAAAGTGCATATGTATATA |
| insert-F1 | AAAGTTTGG-3′ |
| (WT Pxluc) | (SEQ ID NO: 8) |
| Forward Primer | 5′-CATGAATCACAAAGTGCATATGCAACCAACT |
| insert-F2 | GAAAACAAGCAGG-3′ |
| (WT-NS Pxluc) | (SEQ ID NO: 9) |
| Reverse Primer | 5′-GTACAGGTTCTCACTGCCACCGCGATCCCCA |
| insert-R | GCCATGCCC-3′ |
| | (SEQ ID NO: 10) |

TABLE 2

| Components | Volume (μL) |
|---|---|
| 2x KOD FX Neo PCR buffer solution | 25 |
| 2 mM dNTPs | 10 |
| 10 μM Primer insert-F | 1.5 |
| 10 μM Primer insert-R | 1.5 |
| Template DNA (10 ng/μL) | 2.5 |
| KOD FX Neo | 1 |
| PCR grade water | 8.5 |
| Total Volume | 50 |

TABLE 3

| | | | | Number |
|---|---|---|---|---|
| Phase | Temperature | Time | | of cycles |
| Pre-denaturation | 94° C. | 2 | min | 1 |
| Degeneration | 98° C. | 10 | s | 30 |
| Annealing | 60° C. | 30 | s | |
| Extension | 68° C. | 40 | s | |
| Extension | 68° C. | 5 | min | 1 |

The template was digested by adding 0.5 μL of DpnI enzyme to the reaction system and incubating at 37° C. for 3 h, and then the products of about 586 bp and 531 bp were recovered as inserted fragments, i.e., inserts. The gel-recovered fragment of 586 bp was the inserted fragment containing a signal peptide, and the gel-recovered fragment of 531 bp was the inserted fragment without a signal peptide.

### 2. Obtaining Linearized Vector

Using the pCold vector as a template, the vector was linearized by means of PCR to facilitate recombination with the inserted fragment. By using KOD FX neo enzymes, PCR reaction system was prepared and PCR reaction was performed according to the manual thereof.

The sequences of primers used in the PCR reaction are shown in Table 4. The PCR reaction system is shown in Table 5. The reaction conditions are shown in Table 6, and the number of PCR cycles is 30.

TABLE 4

| Name | Sequence |
|---|---|
| Forward Primer | 5′-GGGCATGGCTGGGGATCGCGGTGGCAGTGAG |
| vector-F1 | AACCTGTAC-3′ |
| | (SEQ ID NO: 11) |
| Reverse Primer | 5′-CCAAACTTTTATATACATATGCACTTTGTGA |
| vector-R1 | TTCATGGTG-3′ |
| (WT Pxluc) | (SEQ ID NO: 12) |
| Reverse Primer | 5′-CCTGCTTGTTTTCAGTTGGTTGCATATGCAC |
| vector-R2 | TTTGTGATTCATG-3′ |
| (WT-NS Pxluc) | (SEQ ID NO: 13) |

TABLE 5

| Components | Volume (μL) |
|---|---|
| 2x KOD FX Neo PCR buffer solution | 25 |
| 2 mM dNTPs | 10 |
| 10 μM Primer vector-F | 1.5 |
| 10 μM Primer vector-R | 1.5 |
| pCold (10 ng/μL) | 2.5 |
| KOD FX Neo enzyme | 1 |
| PCR grade water | 8.5 |
| Total Volume | 50 |

TABLE 6

| | | | | Number |
|---|---|---|---|---|
| Phase | Temperature | Time | | of cycles |
| Pre-denaturation | 94° C. | 2 | min | 1 |
| Degeneration | 98° C. | 10 | s | 30 |
| Annealing | 58° C. | 30 | s | |
| Extension | 68° C. | 5 | min | |
| Extension | 68° C. | 5 | min | 1 |

The template was digested by adding 0.5 μL of DpnI enzyme to the reaction system and incubating at 37° C. for 3 h, and then the product of about 4433 bp was recovered as a linearized vector.

Insert and vector were reconstituted using the In-Fusion Cloning kit (TAKARA) according to the reaction system shown in Table 7 and incubated at 50° C. for 15 min.

TABLE 7

| Components | Volume (μL) |
|---|---|
| Inserted fragment (10 ng/μL) | 5 |
| Linear vector (10 ng/μL) | 3 |
| Premix | 2 |
| Total Volume | 10 |

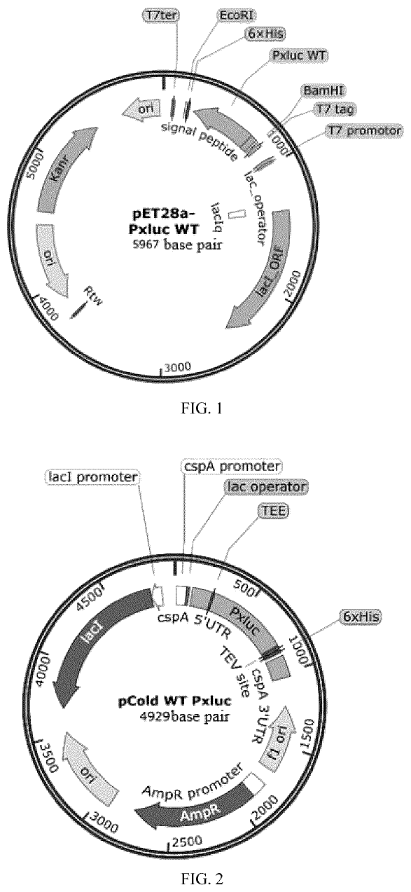

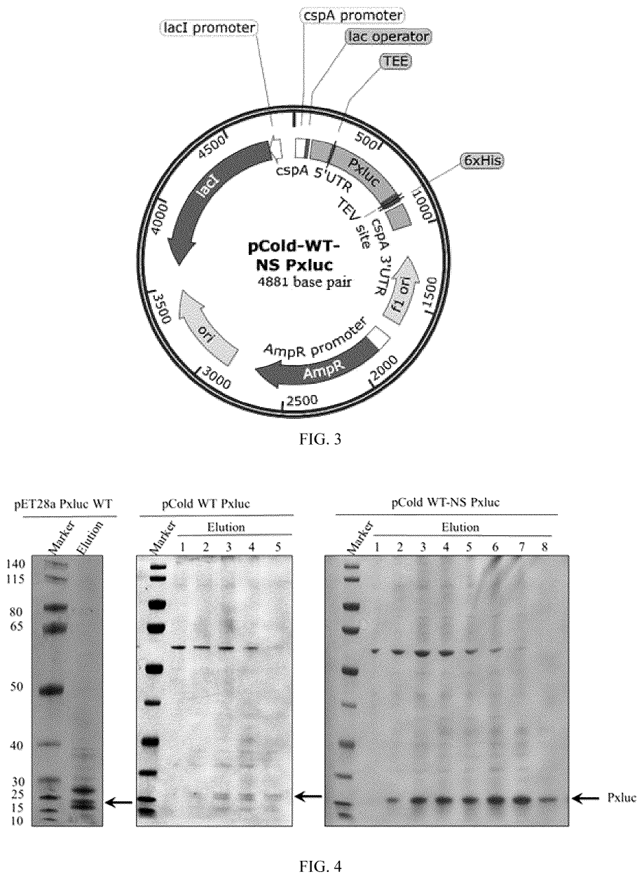

2.5 μL of the above reaction product was taken and transformed into DH5α competent cells and spread onto plates containing ampicillin resistance with a final concentration of 100 μg/mL. On the next day, a single clone was picked from the plates, and the plasmids were extracted for sequencing to ensure that the target fragment was correctly inserted into the vector. The obtained plasmid was the wild Pleuromamma xiphia luciferase containing a signal peptide, i.e., pCold-WT Pxluc (FIG. 2); and the wild Pleuromamma xiphia luciferase without a signal peptide, i.e., pCold WT NS Pxluc (FIG. 3), which were consistent with the experimental expectation.

### Example 2: Prokaryotic Expression, Purification, and Protein Level Activity Assay of Pleuromamma xiphia Luciferase

1. Prokaryotic Expression of Pleuromamma xiphia Luciferase

The expression plasmid pET28a-Pxluc WT was transformed into BL21 (DE3) competent cells, and the expression plasmids pCold WT Pxluc and pCold WT-NS Pxluc were transformed into OrigamiB (DE3) chemically competent cells (EC1020S, WEIDI), respectively. The cells were plated, and a single colony was picked from the plate and incubated overnight at 37° C. On the next day, the cells were diluted at a ratio of 1:100 and transferred to 300 ml of fresh LB medium containing kanamycin (50 μg/ml) or ampicillin (100 μg/ml), respectively. The cells were incubated at 37° C. with shaking at 200 rpm until OD600 was about 0.5 to 0.6, and cooled on ice for 1 h. The inducer IPTG was added at a final concentration of 1 mM and induced overnight at 16° C.

2. Purification of Pleuromamma xiphia Luciferase

The induced bacterial liquid precipitate was collected by centrifugation at a speed of 8,000 rpm/min for 10 min, and added with 30 ml of binding buffer solution (50 mM Tris, 250 mM NaCl, pH 8.0) and 300 μL of lysozyme. The mixture was lysed on ice for 30 min, disrupted by ultrasonic wave (turning on for 2 seconds, turning off for 3 seconds, 60% power) for 30 min, and the supernatant (cell lysate) and precipitate were separated by centrifugation at 12,000 rpm for 30 min at 4° C.

2 mL of HisTrap FF filler was added to the manual column (purchased from Sangon Biotech, model F506607-0001 #affinity chromatography column empty column) and the equilibrated filler was washed with 30 ml of binding buffer solution. Approximately 30 ml of filtered cell lysate was then added. After washing 10 times (10 ml/time) with rinsing solution (50 mM Tris, pH 8.0, 250 mM NaCl, 10 mM imidazole) and then protein was eluted 4 to 5 times with 500 μL of eluent (50 mM Tris, pH 8.0, 250 mM NaCl, 300 mM imidazole), and the eluted protein was collected. Dialysis was performed overnight at 4° C. Protein concentration and purity profiles were determined by the BCA quantification kit method and the SDS-PAGE method.

The protein obtained after elution was determined by the SDS-PAGE method, and the purification results are shown in FIG. 4. FIG. 4 indicates that the target protein was successfully expressed in BL21 (DE3) cells transformed with pET28a-Pxluc WT plasmid, as well as in origamiB (DE3) cells transformed with expression plasmids pCold WT Pxluc and pCold WT-NS Pxluc.

3. Protein Activity Assay for Pleuromamma xiphia Luciferase

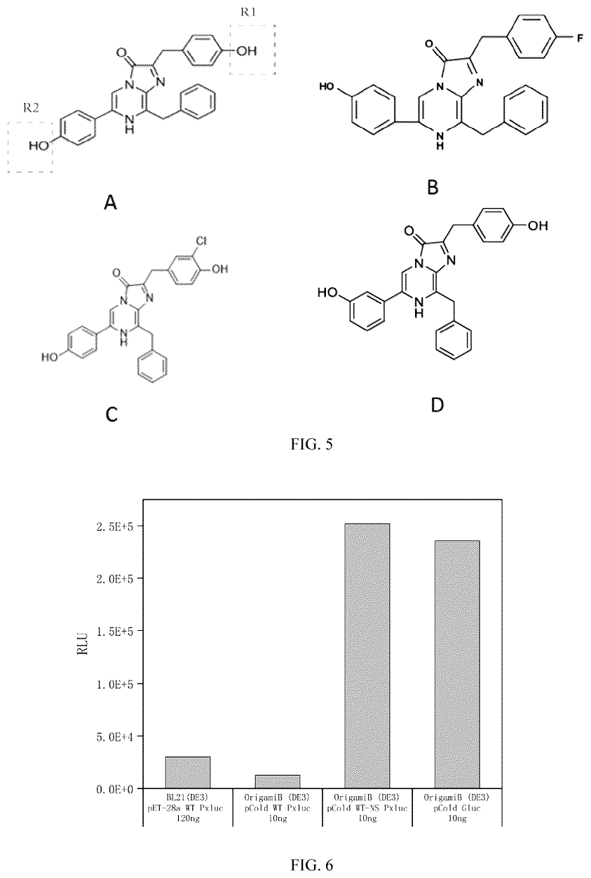

The concentration of protein was accurately determined using a BCA quantification kit (Thermo Scientific™ Pierce™ BCA Protein Assay Kit), and the above purified luciferase was diluted with diluent (50 mM Tris-HCl pH 8.0, 100 mM NaCl, 0.1% (v/v) Tween-20). The protein expressed by the pET28a-Pxluc WT plasmid was diluted to 12 μg/ml, and the proteins expressed by the pCold WT Pxluc plasmid and pCold WT-NS Pxluc plasmid were diluted to 1 g/ml. 10 μL of each protein was added to a black 96-well plate, which was then added with 90 L of the respective substrates, coelenterazine (purchased from Baiaolaibo), fluorinated coelenterazine, and coelenterazine derivatives ZS2, ZS26 (the structural formula of each compound is shown in FIG. 5) that were diluted to 100 M with the same solution, and the luminescence intensity was read from the luminescence module with a microplate reader. The activity assay results of Pleuromamma xiphia luciferase and coelenterazine (CTZ) were shown in FIG. 6. Gluc expressed in OrigamiB (DE3) competent cells was known to have good luminescence activity, and thus it was used as a control group. The results in FIG. 6 show that OrigamiB (DE3) competent cells containing plasmid pCold WT NS Pxluc had the highest luminescence value, which is comparable to that of OrigamiB (DE3) competent cells containing plasmid pCold Gluc. Therefore, the luminescence activity of Pleuromamma xiphia luciferase without a signal peptide to coelenterazine is similar to that of Gluc to coelenterazine. Compared with Pleuromamma xiphia luciferase containing a signal peptide, Pleuromamma xiphia luciferase without a signal peptide had higher activity. Subsequent experiments were performed with Pleuromamma xiphia luciferase without a signal peptide.

### Example 3: Design and Construction of Library of Pleuromamma xiphia Luciferase Mutants

Using pCold WT-NS Pxluc plasmid sequence as a template, site-directed saturation mutant libraries (specific amino acid was mutated to any other amino acid) of sites G98, Q99, G100, and G101 on sequence as set forth in SEQ ID NO: 2 were synthesized by PCR technique. The mutant library 1 was a site-directed saturation mutant library at amino acid position G98 (L1). The mutant library 2 was a site-directed saturation mutant library at site Q99 (L2). The mutant library 3 was a site-directed saturation mutant library at amino acid position G100 (L3). The mutant library 4 was a site-directed saturation mutant library at site G101 (L4). The mutant library 5 was a combination mutant library at amino acid sites G98 and Q99 (L5). The mutant library 6 was a combination mutant library at amino acid sites G100 and G101 (L6). The mutant library 7 was a combination mutant library at amino acid sites G98, Q99, G100 and G101 (L7).

A pair of primers was designed for the mutant library L1:

Forward Primer F1:
(SEQ ID NO: 14)
5′-GATAAATCTATTNNKCAGGGAGGCATAGGTGGCCCTATTG-3′
Reverse Primer R1:
(SEQ ID NO: 15)
5′-TAGGGCCACCTATGCCTCCCTGMNNAATAGATTTATCTCC-3′

A pair of primers was designed for the mutant library L2:

Forward Primer F2:
(SEQ ID NO: 16)
5′-GATAAATCTATTGGANNKGGAGGCATAGGTGGCCCTATTG-3′
Reverse Primer R2:
(SEQ ID NO: 17)
5′-TAGGGCCACCTATGCCTCCMNNTCCAATAGATTTATCTCC-3′

A pair of primers was designed for the mutant library L3:

Forward Primer F3:
(SEQ ID NO: 18)
5′-GATAAATCTATTGGACAGNNKGGCATAGGTGGCCCTATTG-3′
Reverse Primer R3:
(SEQ ID NO: 19)
5′-TAGGGCCACCTATGCCMNNCTGTCCAATAGATTTATCTCC-3′

A pair of primers was designed for the mutant library L4:

Forward Primer F4:
(SEQ ID NO: 20)
5′-GATAAATCTATTGGACAGGGANNKATAGGTGGCCCTATTG-3′
Reverse Primer R4:
(SEQ ID NO: 21)
5′-TAGGGCCACCTATMNNTCCCTGTCCAATAGATTTATCTCC-3′

A pair of primers was designed for the mutant library L5:

Forward Primer F5:
(SEQ ID NO: 22)
5′-GATAAATCTATTNNKNNKGGAGGCATAGGTGGCCCTATTG-3′
Reverse Primer R5:
(SEQ ID NO: 23)
5′-TAGGGCCACCTATGCCTCCMNNMNNAATAGATTTATCTCC-3′

A pair of primers was designed for the mutant library L6:

Forward Primer F6:
(SEQ ID NO: 24)
5′-GATAAATCTATTGGACAGNNKNNKATAGGTGGCCCTATTG-3′
Reverse Primer R6:
(SEQ ID NO: 25)
5′-TAGGGCCACCTATMNNMNNCTGTCCAATAGATTTATCTCC-3′

A pair of primers was designed for the mutant library L7:

Forward Primer F7:
(SEQ ID NO: 26)
5′-GAAGGAGATAAATCTATTNNKNNKNNKNNKATAGGTGGCCCTATTG
TTGATA-3′
Reverse Primer R7:
(SEQ ID NO: 27)
5′-CAATAGGGCCACCTATMNNMNNMNNMNNAATAGATTTATCTCCTTC
ATAAGT-3′

In nucleic acid sequences, “N” is A, C, G, or T; “K” is G or T; and “M” is A or C.

Table 8 shows a reaction system for carrying out the error-prone PCR, and Table 9 shows reaction conditions, with 30 PCR cycles.

TABLE 8

| Components | Volume (μL) |
|---|---|
| 2x KOD FX Neo PCR buffer solution | 25 |
| 2 mM dNTPs | 10 |
| 10 μM Primer F | 1.5 |
| 10 μM Primer R | 1.5 |
| pCold WT-NS Pxluc template DNA (10 ng/μL) | 2.5 |
| KOD FX Neo enzyme | 1 |
| PCR grade water | 8.5 |
| Total Volume | 50 |

TABLE 9

| | | | | Number |
|---|---|---|---|---|
| Phase | Temperature | Time | | of cycles |
| Pre-denaturation | 94° C. | 2 | min | 1 |
| Degeneration | 98° C. | 10 | s | 30 |
| Annealing | 58° C. | 30 | s | |
| Extension | 68° C. | 5 | min | |
| Extension | 68° C. | 5 | min | 1 |

After completion of the reaction, the template was digested by adding 0.5 μL of DpnI enzyme to the reaction system and incubating at 37° C. for 3 h. The digested product was then transformed into DH5a competent cells and 10 ml of the plasmid was extracted overnight culture. After the transformation of the extracted plasmid into OrigamiB (DE3) chemically competent cells (WEIDI, EC1020S), the cells were plated and a single colony was picked from the plate and incubated overnight at 37° C. On the next day, the cells were diluted at a ratio of 1:100 and transferred to 0.4 ml of fresh LB medium containing ampicillin resistance (100 g/ml). The cells were incubated at 37° C. with shaking at 200 rpm until OD600 was about 0.5 to 0.6, and cooled on ice for 1 h. The inducer IPTG was added at a final concentration of 1 mM and induced overnight at 16° C.

50 μL of the induced bacterial solution was added into a black 96-well plate, and then 10 μL of the substrates with a final concentration of 100 PM, i.e., CTZ (purchased from Baiaolaibo), fluorinated coelenterazine, ZS2, and ZS26, were added, respectively. The luminescence intensity was read from the luminescence module with a microplate reader to perform bacterial solution screening of mutants (screening criteria: the screening was performed based on a ratio of the luminescence intensity; when the ratio of the luminescence intensity of substrate ZS2 or ZS26 to the luminescence intensity of substrate F-CTZ is greater than the ratio of the luminescence intensity of wild type to the luminescence intensity of two substrates, it can be regarded as the dominant mutant).

### Example 4: Expression and Purification of Mutant Library of Pleuromamma xiphia Luciferase

1. Prokaryotic Expression of Pleuromamma xiphia Luciferase Mutants

The mutant bacterial solutions screened from the mutant libraries, pCold-no sp Pxluc L1, pCold-no sp Pxluc L2, pCold-no sp Pxluc L3, pCold-no sp Pxluc L4, and pCold-no sp Pxluc L5, were cultured overnight at 37° C., diluted at a ratio of 1:100 on the next day, transferred into 15 ml fresh LB medium containing ampicillin (100 μg/ml), cultured at 37° C. with shaking at 200 rpm until OD600 about 0.5 to 0.6, and cooled on ice for 1 h. The inducer IPTG was added at a final concentration of 1 mM and induced overnight at 16° C.

2. Purification of Pleuromamma xiphia Luciferase Mutants

The induced bacterial liquid precipitate was collected by centrifugation at a speed of 8,000 rpm/min for 10 min, and added with 600 μL of binding buffer solution (50 mM Tris, pH 8.0, 250 mM NaCl) and 6 μL of lysozyme. The mixture was lysed on ice for 30 min, disrupted by ultrasound (turning on for 2 seconds, turning off for 3 seconds, 60% power) for 10 min, and the supernatant (cell lysate) and precipitate were separated by centrifugation at 12,000 rpm for 30 min at 4° C.

50 μL of HisTrap FF filler was added to the manual column (purchased from Sangon Biotech, model F506607-0001 #affinity chromatography column empty column), and the equilibrated filler was washed with 3 ml of binding buffer solution. 600 μL of filtered cell lysate was then added. After washing 10 times (3 ml/time) with rinsing solution (50 mM Tris, pH 8.0, 250 mM NaCl, 10 mM imidazole), the protein was eluted with 100 μL of eluent (50 mM Tris, pH 8.0, 250 mM NaCl, 300 mM imidazole), and the eluted protein was collected.

The protein eluted from the Ni column was dialyzed using dialysis buffer solution (25 mM Tris, pH 8.0, 250 mM NaCl) at 4° C. overnight. Protein concentration and purity profiles were determined by the BCA quantification kit (Thermo Scientific™ Pierce™ BCA Protein Assay Kit) and the SDS-PAGE method.

### Example 5: Substrate Specificity Assay of Dominant Mutants of Pleuromamma xiphia Luciferase

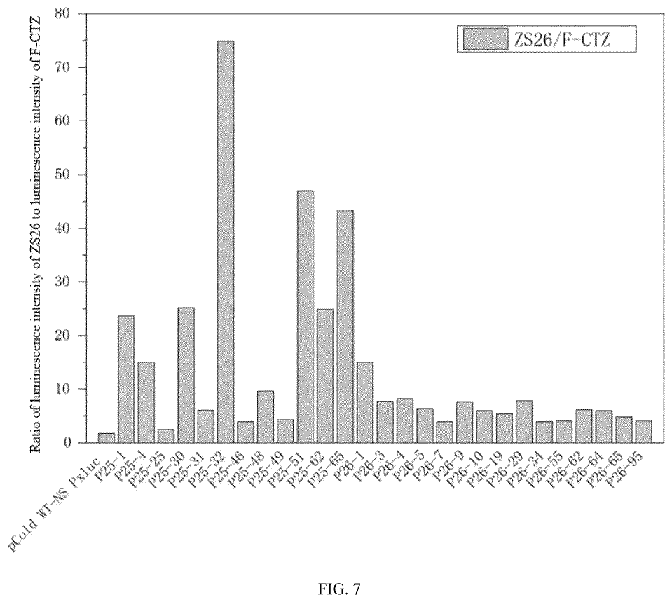

Protein concentration was accurately determined using the BCA quantification kit (Thermo Scientific™ Pierce™ BCA Protein Assay Kit), and luciferase was diluted to 1 μg/ml with diluent (50 mM Tris-HCl pH 8.0, 100 mM NaCl, 0.1% (v/v) Tween-20) and 10 μL was added to black 96-well plates, which was then added with 90 μL of the respective substrates, coelenterazine (purchased from Baiaolaibo), fluorinated coelenterazine, and coelenterazine derivatives ZS2 and ZS26 that were diluted to 100 μM with the same solution, and the luminescence intensity was read from the luminescence module with a microplate reader. The substrate specificity of the mutants was compared with the activity ratio of ZS2/F-CTZ and ZS26/F-CTZ. The activity assay results of the dominant specific mutants are shown in FIG. 7 and FIG. 8. Table 10 below lists results of the mutation sites and substrate specificity of ZS26/F-CTZ and ZS2/F-CTZ of the dominant mutant of Pleuromamma xiphia luciferase.

TABLE 10

| | | $Fold ( ratio of RLU \frac{ZS 2}{F - CTZ} ) ZS 2$ | $Fold ( ratio of RLU \frac{ZS 26}{F - CTZ} ) ZS 26$ |
|---|---|---|---|
| | Mutation site | luminescence intensity to F- | luminescence intensity to |
| No. | of mutant | CTZ luminescence intensity | F-CTZ luminescence intensity |
| pCold WT-NS | Wild type | 3.56 | 1.77 |
| Pxluc | | | |
| P25-1 | G98L, Q99R | 21.808 | 23.647 |
| P25-4 | G98P | 7.476 | 15.032 |
| P25-25 | G98Q | 5.932 | 2.492 |
| P25-30 | G98S, Q99W | 24.750 | 25.221 |
| P25-31 | Q99I | 10.543 | 6.047 |
| P25-32 | Q99Y | 3.841 | 74.857 |
| P25-46 | Q99A | 2.824 | 3.918 |
| P25-48 | Q99L | 2.994 | 9.601 |
| P25-49 | Q99F | 2.022 | 4.259 |
| P25-51 | G98L, Q99V | 23.173 | 46.976 |
| P25-62 | G98T, Q99P | 12.651 | 24.873 |
| P25-65 | G98L, Q99E | 14.841 | 43.355 |
| P26-1 | Q99M, G100S | 3.419 | 15.041 |
| P26-3 | G100Q, G101F | 6.21 | 7.71 |
| P26-4 | G100R, G101R | 5.83 | 8.22 |
| P26-5 | G100W, G101F | 8.16 | 6.36 |
| P26-7 | G100S, | 4.072 | 3.912 |
| P26-9 | G100T, G101S | 6.53 | 7.64 |
| P26-10 | G100R, G101C | 3.88 | 5.98 |
| P26-19 | G100A, G101R | 2.85 | 5.36 |
| P26-29 | G100L, G101Y | 5.20 | 7.83 |
| P26-34 | G100S, G101L | 2.21 | 3.97 |
| P26-55 | G101I | 3.76 | 4.06 |
| P26-62 | G100L, G101K | 3.56 | 6.17 |
| P26-64 | G100T | 3.57 | 5.97 |
| P26-65 | G100A, G101V | 2.89 | 4.79 |
| P26-95 | G100A, G101P | 1.97 | 4.00 |

The results in Table 10 and FIG. 7 and FIG. 8 reveal that: the wild type Pleuromamma xiphia luciferase without a signal peptide had a substrate specificity of about 1.77-fold for ZS26/F-CTZ and about 3.56-fold for ZS2/F-CTZ; the dominant mutant obtained by modifying the enzyme had an increased specificity for the substrate ZS26/F-CTZ, with the highest being about 74.857 times; its specificity for the substrate ZS2/F-CTZ was increased, with the highest being about 24.750 times.

### Example 6: Design and Construction of Pleuromamma xiphia Luciferase Plasmid to be Expressed in Eukaryotic Cells

Pleuromamma xiphia luciferase containing a signal peptide used in the pEE12.4 vector was synthesized by the PCR technique, having the gene sequence as set forth in SEQ ID NO: 28. The signal peptide at N terminal has a histidine tag (6×His) for purification, and the C terminal had an Avitag for biotinylating.

(SEQ ID NO: 28)
ATGTATATAAAAGTTTGGTTTGGTCTGGCTTGTCTTTCATTGGTTCTGGC
CcaccaccaccaccatcacggcagcCAACCAACTGAAAACAAGCAGGAGT
CTCATATTGTAGATTCAGATCTTGATGGCGACCGTGGTAGGAAGTTGCCC
GGAAAAAAGCTTCCTATAGAAGTACTCAAAATCATGGAAGCCAATGCCAG
GAGAGCTGGTTGCACTAGAGGATGTCTCATATGTCTTTCAAAAATCAAGT
GTACAGCCAAAATGAAGCGATACATTCCAGGGAGATGTCATACTTATGAA
GGAGATAAATCTATTGGACAGGGAGGCATAGGTGGCCCTATTGTTGATAT
TCCTGAAATTATTGGATTCAAGAACATGGAACCCATGGATCAGTTCATCG
CACAAGTTGATCTGTGCGCCGACTGTACAACTGGGTGCCTGAAAGGCCTT
GCTAATGTTAGGTGCAATGACTTGCTGAAGAAATGGCTGCCTGACAGATG
TGCTGGTTTTGCCGACAAAATTCAAAATGAAGTGGATAGTATCAAGGGca
tggctggggatcgcggctccggactgaatgatatc

Plasmid for eukaryotic expression of Pleuromamma xiphia luciferase adopted prokaryotic expression plasmid pET28a Pxluc WT containing a signal peptide as the template DNA. The sequences of the primers used in the PCR reaction for constructing the inserted fragment of the expression plasmid are shown in Table 11. The reaction system is shown in Table 12. The reaction conditions are shown in Table 13.

TABLE 11

| Name | Sequence |
|---|---|
| Forward Primer | 5′-CACCACCATCACGGCAGCCAACCAACTGAAAA |
| insert-F | CAAG-3′ |
| | (SEQ ID NO: 29) |
| Reverse Primer | 5′-GATATCATTCAGTCCGGAGCCGCGATCCCCAG |
| insert-R | CCATG-3′ |
| | (SEQ ID NO: 30) |

TABLE 12

| Components | Volume (μL) |
|---|---|
| 2x KOD FX Neo PCR buffer solution | 25 |
| 2 mM dNTPs | 10 |
| 10 μM Primer insert-F | 1.5 |
| 10 μM Primer insert-R | 1.5 |
| Template DNA pET28a Pxluc WT (10 ng/μL) | 2.5 |
| KOD FX Neo enzyme | 1 |
| PCR grade water | 8.5 |
| Total Volume | 50 |

TABLE 13

| | | | | Number |
|---|---|---|---|---|
| Phase | Temperature | Time | | of cycles |
| Pre-denaturation | 94° C. | 2 | min | 1 |
| Degeneration | 98° C. | 10 | s | 30 |
| Annealing | 58° C. | 30 | s | |
| Extension | 68° C. | 5 | min | |
| Extension | 68° C. | 5 | min | 1 |

The template was digested by adding 0.5 μL of DpnI enzyme to the reaction and incubating for 3 h at 37° C., and then the product of about 528 bp was recovered as inserted fragment, i.e., insert.

The vector was linearized by means of PCR using the pEE12.4 vector as a template to facilitate recombination with the inserted fragment. By using KOD FX neo enzymes, PCR reaction system was prepared and PCR reaction was performed according to the manual thereof. The sequences of primers used are shown in Table 14. The PCR reaction system is shown in Table 15. The PCR reaction conditions are shown in Table 16.

TABLE 14

| Name | Sequence (5′-3′) |
|---|---|
| Forward primer | 5′-CATGGCTGGGGATCGCGGCTCCGGACTGAATG |
| | ATATC-3′ |
| | (SEQ ID NO: 31) |
| Reverse primer | 5′-CTTGTTTTCAGTTGGTTGGCTGCCGTGATGGT |
| | GGTG-3′ |
| | (SEQ ID NO: 32) |

TABLE 15

| Components | Volume (μL) |
|---|---|
| 2x KOD FX Neo PCR buffer solution | 25 |
| 2 mM dNTPs | 10 |
| 10 μM Primer vector-F | 1.5 |
| 10 μM Primer vector-R | 1.5 |
| pEE12.4 plasmid (10 ng/μL) | 2.5 |
| KOD FX Neo | 1 |
| PCR grade water | 8.5 |
| Total Volume | 50 |

TABLE 16

| | | | | Number |
|---|---|---|---|---|
| Phase | Temperature | Time | | of cycles |
| Pre-denaturation | 94° C. | 2 | min | 1 |
| Degeneration | 98° C. | 10 | s | 30 |
| Annealing | 58° C. | 30 | s | |
| Extension | 68° C. | 5 | min | |
| Extension | 68° C. | 5 | min | 1 |

The template was digested by adding 0.5 μL of DpnI enzyme to the reaction system and incubating at 37° C. for 3 h, and then the product of about 7712 bp was recovered as a linearized vector.

The insert obtained in the present example was recombined with the vector using the Takara In-Fusion Cloning kit according to the reaction system shown in Table 17, and the reaction condition was 50° C. for 15 min.

TABLE 17

| Components | Volume (μL) |
|---|---|
| insert (10 ng/μL) | 5 |
| vector (10 ng/μL) | 3 |
| Premix | 2 |
| Total Volume | 10 |

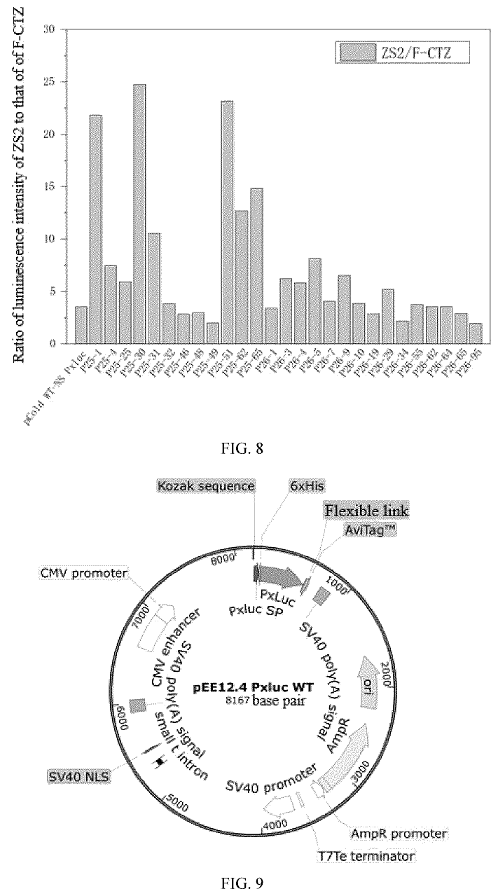

2.5 μL of the above reaction product was transformed into DH5α competent cells and spread onto plates containing ampicillin resistance at a final concentration of 100 μg/mL. On the next day, a single clone was picked from the plates, and plasmids were extracted. Sequencing was performed to ensure that the target fragment was correctly inserted into the vector and that the resulting plasmid was the wild-type luciferase pEE12.4-Pxluc WT for eukaryotic expression. The resulting plasmid was tested and the detected plasmid profile is shown in FIG. 9, which is consistent with the expectation.

The mutant plasmid pEE12.4 Pxluc P26-95 for eukaryotic expression was synthesized by PCR method using the constructed pEE12.4-Pxluc WT as template DNA. The sequences of primers are shown in Table 18. The PCR reaction system is shown in Table 19. The PCR reaction conditions are the same as those in Table 16.

TABLE 18

| Name | Sequence (5′-3′) |
|---|---|
| Forward primer | 5′-GATAAATCTATTGGACAGGCGCCTATAGGTGG |
| | CCCTATTG-3′ |
| | (SEQ ID NO: 33) |
| Reverse primer | 5′-TAGGGCCACCTATAGGCGCCTGTCCAATAGAT |
| | TTATCTCC-3′ |
| | (SEQ ID NO: 34) |

TABLE 19

| Components | Volume (μL) |
|---|---|
| 2x KOD FX Neo PCR buffer solution | 25 |
| 2 mM dNTPs | 10 |
| 10 μM Primer vector-F | 1.5 |
| 10 μM Primer vector-R | 1.5 |
| pEE12.4 Pxluc P26-95 (10 ng/μL) | 2.5 |
| KOD FX Neo | 1 |
| PCR grade water | 8.5 |
| Total Volume | 50 |

After completion of the reaction, the template was digested by adding 0.5 μL of DpnI enzyme to the reaction system, and incubating at 37° C. for 3 h. 2.5 μL of the digested reaction product was transformed into DH5α competent cells and plated on ampicillin-resistant plates containing a final concentration of 100 μg/mL. On the next day, a single clone was picked from the plates, and plasmids were extracted. Sequencing was performed to ensure that the mutant sequence was correct and the resulting plasmid was the mutated luciferase pEE12.4 Pxluc P26-95 (luciferase mutant expression vector containing a signal peptide sequence, mutation sites G100A and G101P) for eukaryotic expression.

### Example 7: Eukaryotic Expression and Protein Purification of Pleuromamma xiphia Luciferase

According to the instructions of ExpiFectamine™ CHO transfection kit (Gibco, A29129), pEE12.4 Pxluc WT and mutant pEE12.4 Pxluc P26-95 plasmids obtained in Example 6 were transfected into 30 mL of Expi-CHO cells, respectively. Seven days after transfection, cell viability was measured less than 90%, and supernatants were collected after centrifugation at 8,000 rpm for 10 min at 4° C.

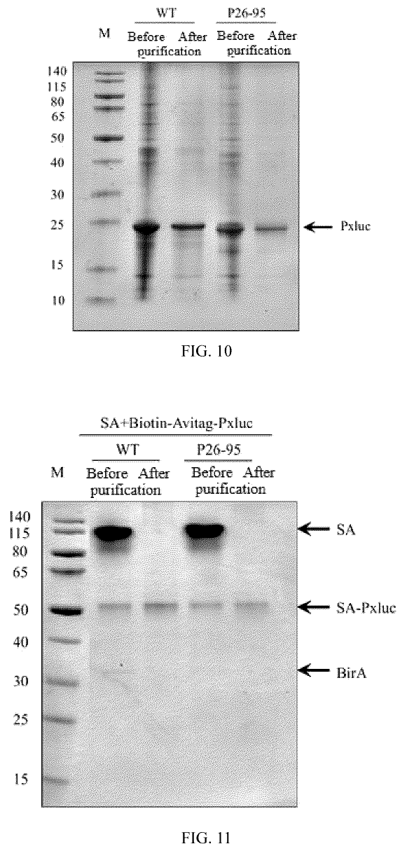

2 mL of HisTrap FF filler was added to the manual column (purchased from Sangon Biotech, model F506607-0001 #affinity chromatography column empty column) respectively and the equilibrated filler was washed with 30 mL of binding buffer solution. Approximately 30 mL of filtered cell supernatant was then added separately. After washing 10 times (10 mL/time) with washing solution (50 mM Tris, pH 8.0, 250 mM NaCl, 10 mM imidazole) and then eluting 4 to 5 times with 500 μL of eluent (50 mM Tris, pH 8.0, 250 mM NaCl, 300 mM imidazole), the eluted proteins were collected separately. The eluted protein was detected by 12% SDS-PAGE, to obtain relatively pure Pxluc protein. The results of purification are shown in FIG. 10.

### Example 8: Coupling and Activity Assay of Dominant Mutants of Pleuromamma xiphia Luciferase

The Pxluc proteins obtained in Example 7, containing the AviTag tag, i.e., AviTag-Pxluc-WT and mutantAviTag-Pxluc-P26-95, were biotinylated as Biotin-Avitag-Pxluc-WT and Biotin-AviTag-Pxluc-P26-95 by BirA enzyme (Avidity, BIRA500) in the reaction system shown in Table 18.

TABLE 20

| Components | Volume (μL) |
|---|---|
| AviTag-Pxluc (1 mg/mL) | 12 |
| BiomixA | 10 |
| BiomixB | 10 |
| BirA (1 mg/mL) | 3 |
| H₂O | 65 |
| Total Volume | 100 |

SA-Pxluc can be prepared by adding SA to the system shown in Table 20, and the histidine tag on Pxluc can be used for further purification to obtain relatively pure SA-Pxluc. The purification results are shown in FIG. 11.

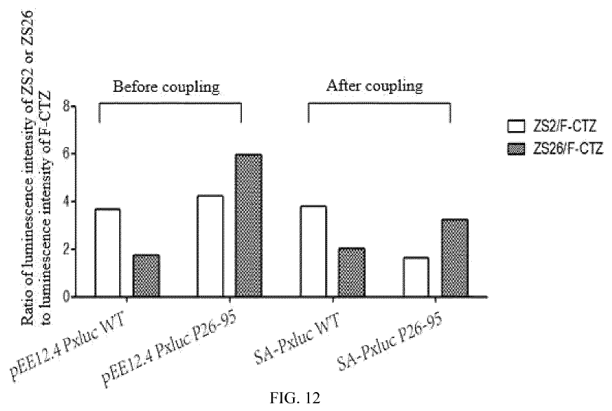

pEE12.4 Pxluc WT and mutant pEE12.4 Pxluc P26-95, as well as the coupled products SA-Pxluc WT and SA-Pxluc P26-95 were subjected to the activity assay according to the method described in Example 5. The results are shown in FIG. 12. FIG. 12 indicates that: both Pxluc WT and mutant Pxluc P26-95 that were expressed in eukaryotic cells had the activity of catalyzing the substrates of coelenterazine derivatives ZS2, ZS26; and compared with Pxluc WT, the mutant Pxluc P26-95 had significantly improved substrate specificity to ZS26/F-CTZ and improved substrate specificity to ZS26/F-CTZ. The substrate specificity of the coupled product SA-Pxluc P26-95 to ZS26/F-CTZ was significantly improved compared with SA-Pxluc WT.

In the specification, references to descriptions of the term “an embodiment”, “some embodiments”, “examples”, “specific examples”, or “some examples”, etc. means that a particular feature, structure, material, or characteristic described in connection with the embodiment or example is included in at least an embodiment or example of the present disclosure. In this specification, schematic representations of the above terms are not necessarily directed to the same embodiment or example. Furthermore, the particular features, structures, materials, or characteristics described may be combined in any suitable manner in one or more embodiments or examples. Furthermore, combinations and combinations of the various embodiments or examples and features of the various embodiments or examples described in this specification can be made by those skilled in the art without departing from the scope of the present disclosure.

While embodiments of the present disclosure have been illustrated and described above, it can be understood that the above-mentioned embodiments are illustrative and not restrictive and that those skilled in the art can make changes, modifications, substitutions, and variations to the above embodiments without departing from the scope of the present disclosure.

What is claimed is:

1. A mutated luciferase, having at least one of the following mutation sites based on the amino acid sequence as set forth in SEQ ID NO: 2: sites 98, 99, 100, and 101, and the mutated luciferase comprising or not comprising a signal peptide amino acid sequence.

2. The mutated luciferase according to claim 1, having one or more of the following mutations (1) to (4) based on the amino acid sequence as set forth in SEQ ID NO: 2:
(1) G at site 98 is mutated to L, P, Q, S, or T;
(2) Q at site 99 is mutated to R, W, I, Y, A, L, F, V, P, E, or M;
(3) G at site 100 is mutated to S, Q, R, W, T, A, or L; and
(4) G at site 101 is mutated to F, R, S, C, Y, L, I, K, V, or P.

3. The mutated luciferase according to claim 1, having one or two of the following mutation sites based on the amino acid sequence as set forth in SEQ ID NO: 2: sites 98, 99, 100 and 101.

4. The mutated luciferase according to claim 3, having one or two of the following mutations (1) to (4) based on the amino acid sequence as set forth in SEQ ID NO: 2:
(1) G at site 98 is mutated to L, P, Q, S, or T;
(2) Q at site 99 is mutated to R, W, I, Y, A, L, F, V, P, E, or M;
(3) G at site 100 is mutated to S, Q, R, W, T, A, or L; and
(4) G at site 101 is mutated to F, R, S, C, Y, L, I, K, V, or P.

5. The mutated luciferase according to claim 1, having the following mutations based on the amino acid sequence as set forth in SEQ ID NO: 2:
1) G at site 98 is mutated to L, and Q at site 99 is mutated to R; or
2) G at site 98 is mutated to P; or
3) G at site 98 is mutated to Q; or
4) G at site 98 is mutated to S, and Q at site 99 is mutated to W; or
5) Q at site 99 is mutated to I; or
6) Q at site 99 is mutated to Y; or
7) Q at site 99 is mutated to A; or
8) Q at site 99 is mutated to L; or
9) Q at site 99 is mutated to F; or
10) G at site 98 is mutated to L, and Q at site 99 is mutated to V; or
11) G at site 98 is mutated to T, and Q at site 99 is mutated to P; or
12) G at site 98 is mutated to L, and Q at site 99 is mutated to E; or
13) Q at site 99 is mutated to M, and G at site 100 is mutated to S; or
14) G at site 100 is mutated to Q, and G at site 101 is mutated to F; or
15) G at site 100 is mutated to R, and G at site 101 is mutated to R; or
16) G at site 100 is mutated to W, and G at site 101 is mutated to F; or
17) G at site 100 is mutated to S; or
18) G at site 100 is mutated to T, and G at site 101 is mutated to S; or
19) G at site 100 is mutated to R, and G at site 101 is mutated to C; or
20) G at site 100 is mutated to A, and G at site 101 is mutated to R; or
21) G at site 100 is mutated to L, and G at site 101 is mutated to Y; or
22) G at site 100 is mutated to S, and G at site 101 is mutated to L; or
23) G mutation at site 101 is I; or
24) G at site 100 is mutated to L, and G at site 101 is mutated to K; or
25) G at site 100 is mutated to T; or
26) G at site 100 is mutated to A, and G at site 101 is mutated to V; or
27) G at site 100 is mutated to A, and G at site 101 is mutated to P.

6. The mutated luciferase according to claim 1, not comprising the signal peptide amino acid sequence.

7. A nucleic acid molecule, encoding the mutated luciferase according to claim 1.

8. An expression vector, comprising the nucleic acid molecule according to claim 7.

9. A recombinant cell, carrying the expression vector according to claim 8.

10. The recombinant cell according to claim 9, wherein the recombinant cell is selected from the group consisting of Escherichia coli, yeast, and mammalian cells.

11. A method for producing a mutated luciferase, comprising:
introducing the expression vector according to claim 8 into recombinant cells;
culturing and propagating the recombinant cells; and
collecting product of said culturing and propagating, and extracting or purifying the mutated luciferase.

12. A method for detecting a nucleic acid sequence using the mutated luciferase according to claim 1, the method comprising:
A) forming, by way of chemical coupling or bioconjugation or by means of fusion protein, a first mutated luciferase complex of a first specific recognition protein and the mutated luciferase, and forming, using a second luciferase as a signal peptide, a second mutated luciferase complex of a second specific recognition protein and the second luciferase by way of chemical coupling or bioconjugation or by means of fusion protein;
B) reacting the first mutated luciferase complex with a first substrate to generate a first luminescent signal, and reacting the second luciferase complex with a second substrate to generate a second luminescent signal; and
C) determining four bases A, T, G, and C for target nucleic acid sequencing by detecting fluorescence signal and signal combination of a self-luminescence system of the mutated luciferase and the second luciferase.

13. The method according to claim 12, wherein:
when the mutated luciferase and the second luciferase are the same, the first substrate and the second substrate are the same; and
when the mutated luciferase and the second luciferase are different, the first substrate and the second substrate are different.

14. A nucleic acid sequencing kit, comprising the mutated luciferase according to claim 1.

15. A method for detecting a content of an analyte, comprising:
a) forming, using the mutated luciferase according to claim 1 as a signal peptide, a complex of a specific recognition protein of the analyte and the mutated luciferase by way of chemical coupling or bioconjugation or by means of fusion protein;
b) contacting the analyte with the complex;
c) adding a substrate for Pleuromamma xiphia luciferase or an analogue of the substrate to the reaction system; and
d) determining the content of the analyte based on a fluorescence intensity of the reaction system detected subsequent to said adding the substrate for the Pleuromamma xiphia luciferase or the analogue of the substrate.

16. The method according to claim 15, wherein the substrate for the Pleuromamma xiphia luciferase is selected from at least one of coelenterazine, fluorinated coelenterazine, or coelenterazine derivative;
optionally, the coelenterazine derivative is selected from coelenterazine derivative ZS2 or coelenterazine derivative ZS26,

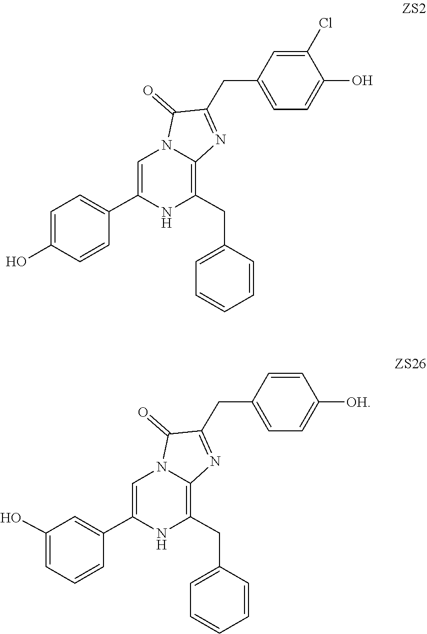 [SMILES: O=c1c(Cc2ccc(O)c(Cl)c2)nc2c(Cc3ccccc3)[nH]c(-c3ccc(O)cc3)cn1-2 ; O=c1c(Cc2ccc(O)cc2)nc2c(Cc3ccccc3)[nH]c(-c3cccc(O)c3)cn1-2]

17. A method for screening a substrate for Pleuromamma xiphia luciferase, comprising:
I) contacting the mutated luciferase according to claim 1 with a substrate to be screened to obtain a reaction mixture; and
II) determining, based on whether a chemical light signal is emitted by the reaction mixture obtained in step I), whether the substrate to be screened is a target substrate.

18. The method according to claim 17, wherein an indicator that the substrate to be screened is the target substrate is that a chemical light signal emitted by the reaction mixture obtained in step I).


SEQUENCE LISTING (34 sequences)

SEQ ID NO: 1 - DNA, 540 bp, synthetic construct
  misc_feature 1..540: note = (WT Pxluc)
  source 1..540: mol_type = other DNA
atgtatataaaagtttggtttggtctggcttgtctttcattggttctggcccaaccaactgaaaacaagcaggagtctcatattgtagattcagatcttgatggcgaccgtggtaggaagttgcccggaaaaaagcttcctatagaagtactcaaaatcatggaagccaatgccaggagagctggttgcactagaggatgtctcatatgtctttcaaaaatcaagtgtacagccaaaatgaagcgatacattccagggagatgtcatacttatgaaggagataaatctattggacagggaggcataggtggccctattgttgatattcctgaaattattggattcaagaacatggaacccatggatcagttcatcgcacaagttgatctgtgcgccgactgtacaactgggtgcctgaaaggccttgctaatgttaggtgcaatgacttgctgaagaaatggctgcctgacagatgtgctggttttgccgacaaaattcaaaatgaagtggatagtatcaagggcatggctggggatcgc

SEQ ID NO: 2 - protein, 180 aa, synthetic construct
  REGION 1..180: note = (WT Pxluc)
MYIKVWFGLACLSLVLAQPTENKQESHIVDSDLDGDRGRKLPGKKLPIEVLKIMEANARRAGCTRGCLICLSKIKCTAKMKRYIPGRCHTYEGDKSIGQGGIGGPIVDIPEIIGFKNMEPMDQFIAQVDLCADCTTGCLKGLANVRCNDLLKKWLPDRCAGFADKIQNEVDSIKGMAGDR

SEQ ID NO: 3 - DNA, 489 bp, synthetic construct
  misc_feature 1..489: note = WT-NS Pxluc
  source 1..489: mol_type = other DNA
caaccaactgaaaacaagcaggagtctcatattgtagattcagatcttgatggcgaccgtggtaggaagttgcccggaaaaaagcttcctatagaagtactcaaaatcatggaagccaatgccaggagagctggttgcactagaggatgtctcatatgtctttcaaaaatcaagtgtacagccaaaatgaagcgatacattccagggagatgtcatacttatgaaggagataaatctattggacagggaggcataggtggccctattgttgatattcctgaaattattggattcaagaacatggaacccatggatcagttcatcgcacaagttgatctgtgcgccgactgtacaactgggtgcctgaaaggccttgctaatgttaggtgcaatgacttgctgaagaaatggctgcctgacagatgtgctggttttgccgacaaaattcaaaatgaagtggatagtatcaagggcatggctggggatcgc

SEQ ID NO: 4 - protein, 163 aa, synthetic construct
  REGION 1..163: note = WT-NS Pxluc
QPTENKQESHIVDSDLDGDRGRKLPGKKLPIEVLKIMEANARRAGCTRGCLICLSKIKCTAKMKRYIPGRCHTYEGDKSIGQGGIGGPIVDIPEIIGFKNMEPMDQFIAQVDLCADCTTGCLKGLANVRCNDLLKKWLPDRCAGFADKIQNEVDSIKGMAGDR

SEQ ID NO: 5 - DNA, 627 bp, synthetic construct
  misc_feature 1..627: note = pET28a pET28a Pxluc WT
  source 1..627: mol_type = other DNA
aagcttgccgccaccatgtatataaaagtttggtttggtctggcttgtctttcattggttctggcccaaccaactgaaaacaagcaggagtctcatattgtagattcagatcttgatggcgaccgtggtaggaagttgcccggaaaaaagcttcctatagaagtactcaaaatcatggaagccaatgccaggagagctggttgcactagaggatgtctcatatgtctttcaaaaatcaagtgtacagccaaaatgaagcgatacattccagggagatgtcatacttatgaaggagataaatctattggacagggaggcataggtggccctattgttgatattcctgaaattattggattcaagaacatggaacccatggatcagttcatcgcacaagttgatctgtgcgccgactgtacaactgggtgcctgaaaggccttgctaatgttaggtgcaatgacttgctgaagaaatggctgcctgacagatgtgctggttttgccgacaaaattcaaaatgaagtggatagtatcaagggcatggctggggatcgcggtggcagtgaaaatctgtattttcagagcggcggccatcatcatcatcatcatggcagctgatgagaattc

SEQ ID NO: 6 - DNA, 586 bp, synthetic construct
  misc_feature 1..586: note = pCold
  source 1..586: mol_type = other DNA
caccatgaatcacaaagtgcatatgatgtatataaaagtttggtttggtctggcttgtctttcattggttctggcccaaccaactgaaaacaagcaggagtctcatattgtagattcagatcttgatggcgaccgtggtaggaagttgcccggaaaaaagcttcctatagaagtactcaaaatcatggaagccaatgccaggagagctggttgcactagaggatgtctcatatgtctttcaaaaatcaagtgtacagccaaaatgaagcgatacattccagggagatgtcatacttatgaaggagataaatctattggacagggaggcataggtggccctattgttgatattcctgaaattattggattcaagaacatggaacccatggatcagttcatcgcacaagttgatctgtgcgccgactgtacaactgggtgcctgaaaggccttgctaatgttaggtgcaatgacttgctgaagaaatggctgcctgacagatgtgctggttttgccgacaaaattcaaaatgaagtggatagtatcaagggcatggctggggatcgcggtggcagtgagaacctgtac

SEQ ID NO: 7 - DNA, 531 bp, synthetic construct
  misc_feature 1..531: note = pCold
  source 1..531: mol_type = other DNA
catgaatcacaaagtgcatatgcaaccaactgaaaacaagcaggagtctcatattgtagattcagatcttgatggcgaccgtggtaggaagttgcccggaaaaaagcttcctatagaagtactcaaaatcatggaagccaatgccaggagagctggttgcactagaggatgtctcatatgtctttcaaaaatcaagtgtacagccaaaatgaagcgatacattccagggagatgtcatacttatgaaggagataaatctattggacagggaggcataggtggccctattgttgatattcctgaaattattggattcaagaacatggaacccatggatcagttcatcgcacaagttgatctgtgcgccgactgtacaactgggtgcctgaaaggccttgctaatgttaggtgcaatgacttgctgaagaaatggctgcctgacagatgtgctggttttgccgacaaaattcaaaatgaagtggatagtatcaagggcatggctggggatcgcggtggcagtgagaacctgta

SEQ ID NO: 8 - DNA, 40 bp, synthetic construct
  misc_feature 1..40: note = Primer insert-F1 WT Pxluc
  source 1..40: mol_type = other DNA
caccatgaatcacaaagtgcatatgtatataaaagtttgg

SEQ ID NO: 9 - DNA, 44 bp, synthetic construct
  misc_feature 1..44: note = Primer insert-F2 (WT-NS Pxluc)
  source 1..44: mol_type = other DNA
catgaatcacaaagtgcatatgcaaccaactgaaaacaagcagg

SEQ ID NO: 10 - DNA, 40 bp, synthetic construct
  misc_feature 1..40: note = Primer insert-R
  source 1..40: mol_type = other DNA
gtacaggttctcactgccaccgcgatccccagccatgccc

SEQ ID NO: 11 - DNA, 40 bp, synthetic construct
  misc_feature 1..40: note = Primer vector-F1
  source 1..40: mol_type = other DNA
gggcatggctggggatcgcggtggcagtgagaacctgtac

SEQ ID NO: 12 - DNA, 40 bp, synthetic construct
  misc_feature 1..40: note = Primer vector-R1 (WT Pxluc)
  source 1..40: mol_type = other DNA
ccaaacttttatatacatatgcactttgtgattcatggtg

SEQ ID NO: 13 - DNA, 44 bp, synthetic construct
  misc_feature 1..44: note = Primer vector-R2 (WT-NS Pxluc)
  source 1..44: mol_type = other DNA
cctgcttgttttcagttggttgcatatgcactttgtgattcatg

SEQ ID NO: 14 - DNA, 40 bp, synthetic construct
  misc_feature 1..40: note = Primer F1
  misc_difference 13..14: note = any of a, c, g, t
  source 1..40: mol_type = other DNA
gataaatctattnnkcagggaggcataggtggccctattg

SEQ ID NO: 15 - DNA, 40 bp, synthetic construct
  misc_feature 1..40: note = Primer R1
  misc_difference 24..25: note = any of a, c, g, t
  source 1..40: mol_type = other DNA
tagggccacctatgcctccctgmnnaatagatttatctcc

SEQ ID NO: 16 - DNA, 40 bp, synthetic construct
  misc_feature 1..40: note = Primer F2
  misc_difference 16..17: note = any of a, c, g, t
  source 1..40: mol_type = other DNA
gataaatctattggannkggaggcataggtggccctattg

SEQ ID NO: 17 - DNA, 40 bp, synthetic construct
  misc_feature 1..40: note = Primer R2
  misc_difference 21..22: note = any of a, c, g, t
  source 1..40: mol_type = other DNA
tagggccacctatgcctccmnntccaatagatttatctcc

SEQ ID NO: 18 - DNA, 40 bp, synthetic construct
  misc_feature 1..40: note = Primer F3
  misc_difference 19..20: note = any of a, c, g, t
  source 1..40: mol_type = other DNA
gataaatctattggacagnnkggcataggtggccctattg

SEQ ID NO: 19 - DNA, 40 bp, synthetic construct
  misc_feature 1..40: note = Primer R3
  misc_difference 18..19: note = any of a, c, g, t
  source 1..40: mol_type = other DNA
tagggccacctatgccmnnctgtccaatagatttatctcc

SEQ ID NO: 20 - DNA, 40 bp, synthetic construct
  misc_feature 1..40: note = Primer F4
  misc_difference 22..23: note = any of a, c, g, t
  source 1..40: mol_type = other DNA
gataaatctattggacagggannkataggtggccctattg

SEQ ID NO: 21 - DNA, 40 bp, synthetic construct
  misc_feature 1..40: note = Primer R4
  misc_difference 15..16: note = any of a, c, g, t
  source 1..40: mol_type = other DNA
tagggccacctatmnntccctgtccaatagatttatctcc

SEQ ID NO: 22 - DNA, 40 bp, synthetic construct
  misc_feature 1..40: note = Primer F5
  misc_difference 13..14: note = any of a, c, g, t
  misc_difference 16..17: note = any of a, c, g, t
  source 1..40: mol_type = other DNA
gataaatctattnnknnkggaggcataggtggccctattg

SEQ ID NO: 23 - DNA, 40 bp, synthetic construct
  misc_feature 1..40: note = Primer R5
  misc_difference 21..22: note = any of a, c, g, t
  misc_difference 24..25: note = any of a, c, g, t
  source 1..40: mol_type = other DNA
tagggccacctatgcctccmnnmnnaatagatttatctcc

SEQ ID NO: 24 - DNA, 40 bp, synthetic construct
  misc_feature 1..40: note = Primer F6
  misc_difference 19..20: note = any of a, c, g, t
  misc_difference 22..23: note = any of a, c, g, t
  source 1..40: mol_type = other DNA
gataaatctattggacagnnknnkataggtggccctattg

SEQ ID NO: 25 - DNA, 40 bp, synthetic construct
  misc_feature 1..40: note = Primer R6
  misc_difference 15..16: note = any of a, c, g, t
  misc_difference 18..19: note = any of a, c, g, t
  source 1..40: mol_type = other DNA
tagggccacctatmnnmnnctgtccaatagatttatctcc

SEQ ID NO: 26 - DNA, 52 bp, synthetic construct
  misc_feature 1..52: note = Primer F7
  misc_difference 19..20: note = any of a, c, g, t
  misc_difference 22..23: note = any of a, c, g, t
  misc_difference 25..26: note = any of a, c, g, t
  misc_difference 28..29: note = any of a, c, g, t
  source 1..52: mol_type = other DNA
gaaggagataaatctattnnknnknnknnkataggtggccctattgttgata

SEQ ID NO: 27 - DNA, 52 bp, synthetic construct
  misc_feature 1..52: note = Primer R7
  misc_difference 18..19: note = any of a, c, g, t
  misc_difference 21..22: note = any of a, c, g, t
  misc_difference 24..25: note = any of a, c, g, t
  misc_difference 27..28: note = any of a, c, g, t
  source 1..52: mol_type = other DNA
caatagggccacctatmnnmnnmnnmnnaatagatttatctccttcataagt

SEQ ID NO: 28 - DNA, 585 bp, synthetic construct
  misc_feature 1..585: note = pEE12.4
  source 1..585: mol_type = other DNA
atgtatataaaagtttggtttggtctggcttgtctttcattggttctggcccaccaccaccaccatcacggcagccaaccaactgaaaacaagcaggagtctcatattgtagattcagatcttgatggcgaccgtggtaggaagttgcccggaaaaaagcttcctatagaagtactcaaaatcatggaagccaatgccaggagagctggttgcactagaggatgtctcatatgtctttcaaaaatcaagtgtacagccaaaatgaagcgatacattccagggagatgtcatacttatgaaggagataaatctattggacagggaggcataggtggccctattgttgatattcctgaaattattggattcaagaacatggaacccatggatcagttcatcgcacaagttgatctgtgcgccgactgtacaactgggtgcctgaaaggccttgctaatgttaggtgcaatgacttgctgaagaaatggctgcctgacagatgtgctggttttgccgacaaaattcaaaatgaagtggatagtatcaagggcatggctggggatcgcggctccggactgaatgatatc

SEQ ID NO: 29 - DNA, 36 bp, synthetic construct
  misc_feature 1..36: note = Primer insert-F
  source 1..36: mol_type = other DNA
caccaccatcacggcagccaaccaactgaaaacaag

SEQ ID NO: 30 - DNA, 37 bp, synthetic construct
  misc_feature 1..37: note = Primer insert-R
  source 1..37: mol_type = other DNA
gatatcattcagtccggagccgcgatccccagccatg

SEQ ID NO: 31 - DNA, 37 bp, synthetic construct
  source 1..37: mol_type = other DNA
catggctggggatcgcggctccggactgaatgatatc

SEQ ID NO: 32 - DNA, 36 bp, synthetic construct
  source 1..36: mol_type = other DNA
cttgttttcagttggttggctgccgtgatggtggtg

SEQ ID NO: 33 - DNA, 40 bp, synthetic construct
  source 1..40: mol_type = other DNA
gataaatctattggacaggcgcctataggtggccctattg

SEQ ID NO: 34 - DNA, 40 bp, synthetic construct
  source 1..40: mol_type = other DNA
tagggccacctataggcgcctgtccaatagatttatctcc